(19) United States
(12) Patent Application Publication
(10) Pub. No.: US 2024/0150714 A1
(43) Pub. Date: May 9, 2024

(54) DEVICE FOR RECOVERING MAGNETICALLY TAGGED TARGET CELLS

(71) Applicant: THE GOVERNING COUNCIL OF THE UNIVERSITY OF TORONTO, Toronto (CA)

(72) Inventors: Shana Olwyn KELLEY, Glencoe, IL (US); Zongjie WANG, Chicago, IL (US); Sharif AHMED, Evanston, IL (US)

(21) Appl. No.: 18/547,832

(22) PCT Filed: Feb. 25, 2022
(86) PCT No.: PCT/CA2022/050274

§ 371 Date: Aug. 24, 2023

Related U.S. Application Data
(60) Provisional application No. 63/153,456, filed on Feb. 25, 2021.
(60) Provisional application No. 63/183,350, filed on May 3, 2021.

(51) Int. Cl. C12N 5/0783; C12M 1/00; C12M 1/12; C12M 1/42; C12M 3/00; C12M 3/06

(52) U.S. Cl. CPC C12N 5/0636; C12M 23/16; C12M 23/44; C12M 25/04; C12M 35/02; C12M 47/04

(57) ABSTRACT

There is provided a device, system and method for recovering magnetically tagged target cells from a fluid collection of cells. The device comprises a magnet plate having an array of micro-magnets and a modular chip releasably coupled to the magnet plate. The modular chip has a flow chamber and a plurality of flow rate-reducing structures. The magnetic field produced by the array of micro-magnets cooperates with the flow rate-reducing structures to define a capture zone in the vicinity of each structure. The magnetic field is sufficient to overcome drag force on the target cells to promote capture of the target cells in the capture zone. Separation of the modular chip from the magnet plate allows for recovery of the captured target cells from the modular chip.

### FIELD

The present disclosure relates generally to devices for recovering cells a collection of cells. In particular, the present disclosure relates to devices that use magnetism for recovering magnetically tagged target cells in a flow chamber.

### BACKGROUND

Tumor-infiltrating lymphocytes (TILs) are a subset of white blood cells that have left the circulation system and migrated into tumor tissue.¹ TILs are implicated in tumor killing process and the presence of TILs in tumors is often associated with better clinical outcomes after therapy.² Because of their potency in killing tumors, TILs have been successfully used for adoptive cell therapy (ACT) to treat cancer and achieved remarkable success in managing highly immunogenic tumors, particularly melanoma.

Despite success in the clinic, prolonged turnaround time significantly limits the application of TIL-based ACT. So far, a typical lead time of TIL-based ACT varies from 6-14 weeks,³ where the growth and expansion of TILs occupy 80% of the lead time.⁴⁻⁶ This approach to generating therapeutic doses of TILs dramatically increases the total cost of TIL-based ACT to >$85,000 per patient.⁷ It is well accepted that more effective TIL isolation/expansion could greatly benefit its practicality and clinical adoption.⁸ Moreover, about 20% of patients clinically deteriorate before completion of TIL manufacturing.⁹ A faster TIL manufacturing process could potentially provide better outcomes for these patients. In addition, several studies suggest that prolonged expansion could alter the phenotype and potency of TILs.^{10,11} TILs that underwent minimal culture and selection in vitro have exhibited a higher level of antigen reactivity and activation when used in vivo.¹² Taken together, it is highly desirable to establish methods that efficiently generate large numbers of highly potent TILs from the original source, and minimizes post isolation processing.

The quantity of TILs after expansion relies on two major factors: the expansion rate and the initial quantity of the cells. While significant effort been put towards to optimize the expanding condition of TILs,^{13,14} very limited work has been done to increase the initial quantity of TILs isolated from a tumor. Enrichment of a TIL subpopulation, such as CD8⁺, could improve the reactivity¹⁵ and specificity¹⁶ of TILs since CD8⁺ TILs are the primary drivers of tumor rejection in patients.¹⁷ In research studies, the enrichment of TILs from digested tumor tissues has been achieved with fluorescence-activated cell sorting (FACS)¹⁸⁻²⁰ or magnetic-activated cell sorting (MACS), but neither of these approaches has been used to isolate TILs for clinical applications.^{21,22} FACS often loses 50-70% of cells due to poor droplet formation or scanning errors.^{23,24} This low level of recovery results in a significant loss of TILs and is likely to hamper the downstream expansion process. Besides, although MACS has better recovery than FACS,²⁴ this column-based approach traps dead cells and debris²⁵ leading to the poor enrichment of small cells including leukocytes.^{26,27}

Microfluidic-based approaches have been used for cell sorting with high specificity and sensitivity.^{28,29} It has been widely applied to the isolation, recovery, and analysis of various mammalian cells such as circulating tumor cells,^{30,31} antigen-specific T cells,^{32,33} and contaminating tumorigenic cells.³⁴ However, existing platforms are not suited for TIL isolation, which requires volumetric, high-recovery, high-purity cell sorting at a reasonable cost.

### SUMMARY

In some examples, the present disclosure describes a device for recovering of magnetically tagged target cells from a fluid collection of cells, the device comprising: a magnet plate having an array of micro-magnets positioned therein, the array of micro-magnets producing a magnetic field along the magnet plate; a modular chip releasably coupled in covering relation to the magnet plate, the modular chip having: a flow chamber with an inlet and an outlet; and a plurality of flow rate-reducing structures in the flow chamber, each structure comprising a trapping surface shaped to reduce flow rate in a vicinity of the trapping surface, the magnetic field produced by the array of micromagnets cooperating with the flow rate-reducing structure to define a respective capture zone in the vicinity of each of the flow rate-reducing structures; wherein the magnetic field, in the capture zone, is sufficiently high to overcome drag force on the target cells to promote capture of the target cells, from the collection of cells, in the capture zone; and wherein separation of the modular chip from the magnet plate allows for separation and recovery of the captured target cells from the modular chip.

In some examples, the present disclosure describes a system for recovering magnetically tagged target cells from a collection of cells, the system comprising: a magnet plate having an array of micro-magnets positioned therein, the array of micro-magnets producing a magnetic field along the magnet plate; one or more modular chips releasably coupled in covering relation to the magnet plate, each of the one or more modular chips having: a flow chamber with an inlet and an outlet; and a plurality of flow rate-reducing structures in the flow chamber, each structure comprising a trapping surface shaped to reduce flow rate in a vicinity of the trapping surface, the magnetic field produced by the array of micro-magnets cooperating with the flow rate-reducing structures to define a respective capture zone in the vicinity of each of the flow rate-reducing structures; wherein the magnetic field, in the capture zone, is sufficiently high to overcome drag force on the target cells to promote capture of the target cells, from the collection of cells, in the capture zone; and wherein separation of the modular chip from the magnet plate allows for separation and recovery of the captured target cells from the modular chip; and a scaffold configured to retain the magnet plate.

In some examples, the present disclosure describes a method for recovering magnetically tagged target cells from a fluid collection of cells, the method comprising: introducing the fluid collection of cells containing the magnetically tagged target cells into a device comprising a magnet plate having an array of micro-magnets positioned therein, the array of micro-magnets producing a magnetic field along the magnet plate, and a modular chip releasably coupled in covering relation to the magnet plate, the modular chip having a flow chamber with a plurality of flow rate-reducing structures, the magnetically tagged target cells being susceptible to a magnetic attraction force and being trapped by the flow rate-reducing structures as they travel through the flow chamber; washing non-target cells out of the device; separating the modular chip from the magnet plate; and recovering the magnetically tagged target cells from the modular chip.

In some examples, the present disclosure describes a method for recovering magnetically tagged target cells from a fluid collection of cells, the method comprising: introducing the fluid collection of cells containing the magnetically tagged target cells into the system as described herein through the inlet of the first modular chip, directing the fluid sample from the outlet of the first modular chip into the inlet of the second modular chip; washing non-target cells out of the system; separating the first and second modular chips from the magnet plate; and recovering the magnetically tagged target cells from the first and second modular chips.

In some examples, the present disclosure describes a method for recovering magnetically tagged target cells from a fluid collection of cells, the method comprising: introducing the fluid collection of cells containing the magnetically tagged target cells into the system as described herein through the inlet of the first modular chip and the inlet of the second modular chip; washing non-target cells out of the system; separating the first and second modular chips from the magnet plate; and recovering the magnetically tagged target cells from the first and second modular chips.

The ability of the modular chip to separate from the magnet plate helps improve throughput in the disclosed devices, systems, and methods and increases the ease in removing cells from the modular chip after sorting. The releasable coupling also allows the system to be field-programmable, i.e. allows for flexibility in changing the configuration for different sorting applications and to make it fit-for-purpose. For example, the releasable coupling of the modular chip to the magnet plate allows the system to be interchangeable between a series configuration and a parallel configuration by the end user. The fact that the modular chip and the magnet plate are separate components also allows for the modular chip to be fabricated more quickly and cheaply using 3D printing and/or injection moulding, instead of using conventional lithography. This may also be beneficial in enabling a hygienic, disposable system, in which a low cost, high volume 3D printing process may be used to fabricate disposable modular chips, which may be particularly desirable in a healthcare setting.

### BRIEF DESCRIPTION OF THE DRAWINGS

Reference will now be made, by way of example, to the accompanying drawings which show example embodiments of the present application, and in which:

FIG. 1 are illustrative drawings of disassembled example devices with different channel thicknesses for magnetic recovery of target cells from a collection of cells.

FIG. 2a is an illustrative drawing of assembled devices of FIG. 1 connected in parallel in a system for magnetic recovery of target cells from a collection of cells according to an embodiment of the present disclosure.

FIG. 2b is an illustrative drawing of assembled devices of FIG. 1 connected in series in a system according to another embodiment of the present disclosure.

FIG. 3 are photographs of the disassembled example devices of FIG. 1.

FIG. 4 is a photograph of the assembled system of FIG. 2a.

FIG. 5a is a photograph of the assembled devices of FIG. 3 according to other embodiments of the present disclosure.

FIG. 5b is an enlarged view of portion A of FIG. 5a.

FIG. 6 are enlarged perspective views of a portion of the X's in each of the devices in FIG. 5a.

FIG. 7 is a figural overview of an example method for recovery of magnetically tagged target cells from a tumor sample for use in adoptive cell therapy.

FIG. 8 is a flowchart setting forth the steps of an example method for recovering target cells from a collection of cells using the devices of FIG. 2a or 2b.

FIG. 9 illustrates a simulated flow velocity distribution within the devices with different heights, unit of color bar (mm·s⁻¹), from FIGS. 5a and 6.

FIG. 10 illustrates quantitation of the simulated flow velocity of FIG. 9.

FIG. 11 is a figural illustration of how magnetically tagged target cells may be separated from non-target cells.

FIG. 12 is a flowchart setting forth the steps of an example method for recovering target cells from a collection of cells using the systems of FIG. 2a or 2b.

FIG. 13a is a figural illustration of the system of FIG. 2a used to recover magnetically tagged target TILs.

FIG. 13b is a figural illustration of the system of FIG. 2b used to recover a subset, CD8⁺ TILs, of the recovered TILs from FIG. 13a.

FIGS. 14a, 14b, and 14c illustrate comparison between binary microfluidic cells sorting and commercialized column-based magnetic cell sorting (MACS). (14a) Representative CD45 profile of K562 and MDA-MB-231 cells used for positive and negative enrichment. (14b) Quantitation of capture performance during positive enrichment. Legends indicate the cell number used for each run. (14c) Quantitation of capture performance during negative enrichment. Legends indicate the cell number used for each run.

FIGS. 15a, 15b, and 15c illustrate comparison between quantitative microfluidic cell sorting and commercialized column-based magnetic cell sorting (MACS). (15a) Representative CD326 profile of PC-3M, MDA-MB-231 and U937 cells. (15b) Quantitation of capture profile of quantitative microfluidic cell sorting and the putative capture cut-off. (15c) Quantitation of capture profile of binary MACS sorting and its putative capture cut-off.

FIGS. 16a, 16b, 16c, and 17 illustrate optimization of flow rate for sorting TILs based on CD4 or CD8, where (16a) is a representative flow cytometric profile of pure human CD4 T cells, pure MDA-MB-231 cells and 1% CD4 T cells spiked in MDA-MB-231 cells. CD4 was occupied by MNPs so CD45 conjugated with APC was used to confirm purity; where (16b) is a representative flow cytometric profile of purified spiked-in samples under different flow rates. Optimal flow rate for human CD4 is 32 mL/hr; where (16c) is a representative flow cytometric profile of purified spiked-in samples under different flow rates. Optimal flow rate for mouse CD8 is 16 mL/hr; and where (17) is a quantitative comparison among the purity of FACS/MACS/MAGIC for isolating 0.1% and 1% spike-in samples (human CD4 in MDA-MB-231 and mouse CD8 in B16F10).

FIGS. 18 and 19 are representative cytometric histograms and quantitation of recovery efficiency of T cell populations purified from 0.1% and 1% spike-in samples (human CD4 in MDA-MB-231 and mouse CD8 in B16F10).

FIG. 20 are pie graphs comparing the cell stress induced by different sorting techniques.

FIG. 21 illustrates a workflow of the mouse melanoma model for TIL isolation.

FIGS. 22 and 23 are representative cytometric plots and quantitation of the purity and recovery rate of TILs on D4 after isolation. TILs were defined as the CD8+/CD45+ population. Pure B16 cells and CD8+ splenocytes were used as negative and positive control. Analysis was performed at D4 to allow the CD8-MNP to degrade and re-expose the CD8 epitopes for fluorescent labelling.

FIG. 24 are graphs illustrating the determination of unique clonotypes and Shannon diversity index within TIL populations based on CDR3 expression using bulk TCR sequencing.

FIG. 25 are graphs illustrating quantitation of the recovery of FACS/MACS/FACS for isolating TILs from human NSCLC patients.

FIG. 26 is a graph illustrating expansion curve of TILs isolated by different methods from the standard counting using hemocytometer.

FIG. 27 illustrates a representative CD45RA/CCR7 profile of MAGIC-TILs after 14 days of expansion.

FIG. 28 illustrates Quantitation of relative mRNA expression of TILs compared to CD8 T cells in blood, using TaqMan microarrays. Upregulation in proliferation, activation, cytotoxicity and immune response-related pathway was observed (NFkB and JAK-STAT). MAGIC TILs have a more significant upregulation of core genes of immune response, such as IFNG, GZMB, NFKB family and STAT family, compared to MACS/FACS TILs.

FIG. 29 is a graph illustrating pathway enrichment using gene sets with log 2(FC)>1.5. It is very likely that positive regulation occurred in T cell proliferation, activation, cytotoxicity, as well as NFkB, JAK-STAT, and TNF pathways. Higher (−log 10(P) score means higher chance of the corresponding pathway to be involved.

FIG. 30 is a graph illustrating cytotoxicity of TILs against B16F10^{OVA-GFP} cells in vitro. MAGIC TILs have higher killing efficacy in vitro.

FIG. 31 is cytokine profile of the supernatant collected from in vitro killing assay. Compared to non-TIL controls, the presence of TILs promotes the secretion of several cytokines/chemokines involved in antigen presenting, monocyte recruitment and anti-angiogenesis. Similar to RNA level, a more significant secretion of IFN was observed in MAGIC TILs compared to MACS/FACS TILs.

FIG. 32 illustrates a Workflow of the scenario 1 study comparing the therapeutic efficacy of TILs expanded for 14 days. Number of available TILs on D14 is 2×10⁶, 5×10⁵ and 5×10⁴ for MAGIC, MACS and FACS per mouse.

FIG. 33 shows representative tumor size of each group on D18.

FIG. 34 are graphs illustrating quantitation of tumor size and survival curve treated by the TILs isolated by MAGIC, MACS and FACS (n=5). (Maximal number of available TILs was injected for each group). Log-rank test was used to determine the statistical significance.

FIG. 35 are graphs illustrating quantitation of tumor size and survival curve treated by the MAGIC-TILs under different doses (n=5). Log-rank test was used to determine the statistical significance.

FIG. 36 are graphs illustrating comparison of the therapeutic efficacy of MAGIC and MACS TILs under the same dose (5×10⁵, n=5) and the therapeutic efficacy of MAGIC and FACS TILs under the same dose (5×10⁴, n=5).

FIG. 37 illustrates workflow of the scenario 2 study comparing the therapeutic efficacy of TILs isolated by different methods, at the optimal dosage ˜5×10⁵ at its earliest (D5 for MAGIC, D10 for MACS, D15 for FACS, for FACS, lower number (5×10⁴) of TILs were injected as it fails to reach desired concentration before mouse of mice developed large tumors).

FIG. 38 shows representative tumor size of each group on D18.

FIG. 39 are graphs illustrating quantitation of tumor size and survival curve treated by the TILs isolated by MAGIC, MACS and FACS (n=5, **p<0.01). Log-rank test was used to determine the statistical significance.

FIG. 40 are representative images of infiltrated T cells in solid tumors (Blue: nuclei, Red: CD8α, Brown: melanin) and quantitation of the number/area coverage of CD8⁺ TILs in the tumors treated by MAGIC, MACS and FACS TILs (n=3, 5 slices per tumor).

FIG. 41 illustrates a workflow of the mouse colon cancer model MC-38 for quantitative sorting based on CD39 in CD8+ TILs.

FIG. 42 show representative cytometric profiles of CD39 expression in bulk CD8+ TIL populations and the characterization of sorted cells from CD39-mediated quantitative sorting.

FIG. 43 illustrates quantitation of relative mRNA expression of different CD39 populations compared to bulk CD8+ TILs.

FIG. 44 are representative cytometric profile and quantitation of cell proliferation based on Ki67 expression.

FIG. 45 are graphs illustrating cytotoxicity of TILs against MC-38 cells in vitro.

FIG. 46 illustrates a workflow of the animal study comparing the therapeutic efficacy of different populations of TILs isolated by quantitative MAGIC based on CD39 expression.

FIG. 47 illustrates representative tumor size of each group on D21.

FIG. 48 are graphs illustrating quantitation of tumor size treated by the TILs with different CD39 expression (n=5-10). Log-rank test was used to determine the statistical significance.

FIG. 49 are graphs illustrating quantitation of tumor size treated by the TILs with different degree of purification (Unsorted, sorted by CD8, sorted by CD8 and CD39, n=5-10). Log-rank test was used to determine the statistical significance.

FIG. 50a illustrate experimental validation of proposed working principle of configurable microfluidic cell sorting, the experiments were carried out using K562 cells labelled by anti-CD45 MNPs and stained by Calcein AM. The images are representative microscopic images of the device with different capture settings. Only the combination of magnetic labelled cells and proper external magnets resulted in efficient cell capture at desired capture pocket.

FIG. 50b are representative microscopic images of the device at different stages of operations. After the removal magnets, nearly 100% of the captured cells could be recovered from the chips, which granted the excellent recovery efficiency during cell sorting.

FIGS. 51, 52, 53, and 54 illustrate isolation and clonotyping of CD8+ TILs in B16F10 melanoma model, where (49) illustrates representative gating strategy used for the FACS isolation of CD8⁺ TILs; where (50) illustrates usage of V and J genes from CD8+ TILs isolated by different approaches. Similar enrichment of genes were found in all approaches, indicating the similar origin and phenotypes of isolated TILs; where (51) illustrates VJ paring conditions of CD8+ TILs isolated by different approaches; and where (52) illustrates quantitation of VJ pairing conditions. High-recovery approach could isolate rare clones of the TILs and therefore better preserved their complexity and diversity;

FIGS. 55 and 56 illustrate comparison of different sorting techniques for human NSCLC samples, where (53) show representative cytometric profiles pre and post-sorting; and (54) illustrate quantitation of the purity of FACS/MACS/FACS.

FIG. 57 illustrates relative expression, where the upper diagrams shows pathway enrichment and the bottom diagram shows immune-related genes by specific pathways. Activated CD8⁺ T cells from spleen was used as an internal reference.

FIGS. 58a, 58b, 59a, and 59b illustrate quantitation of the CD8⁺ TILs in the tumors underwent adoptive cell therapy, where (58a) shows random forest-based tumor classifier was trained by user-defined tumor/stroma/glass regions. The trained classifier was then applied to perform whole-slide segmentation to identify tumor. Stroma and glass regions were excluded in downstream analysis; where (58b) shows overlaid bright-field images were decomposed by a CytoNuclear algorithm to reconstruct channels of hematoxylin (blue), warp red and brown. Decomposed images were used to calculate the positive area of each channel by thresholding; where (59a) shows cell number of CD8⁺ TILs were quantified by automated cell counting algorithm using decomposed images. TILs were defined as hematoxylin⁺/warp red⁺; and where (59b) shows representative decomposed images from the tumors underwent adoptive cell therapy.

FIGS. 60a, 60b, and 61 illustrate flow cytometric analysis of TILs from different CD39 populations in a MC-38 mouse model, where (60a) is a MC-38 model has about 1% CD8+ TILs within the tumors according to CD8/CD45 gating; where (60b) is a representative cytometric profile and quantitation of exhaustion markers (PD-1, TIM3, TIGIT); and where (61) is a representative cytometric profile and quantitation of intracellular cytokines (IFN, TNF, IL-2).

FIG. 62a illustrates benchmarking CD326 cross different cancer cell lines. A mixture of these cell lines was generated to have a fairly uniform and broad spectrum of CD326 expression as the input. FIG. 62b is a representative flow cytometric profile and median fluorescence intensity (MFI) of the population recovered from each module running at the flow rate of 16 mL/hr. FIG. 62c provides the MFI of the population recovered from each module running at the flow rate of 8 mL/hr.

FIG. 63 shows characterization of the capability of a 3D printer for printing a mold for fabricating an example of the disclosed modular chip. The 3D printer is shown to better support the shape formation of positive structures (i.e. microposts) than the negative structures (i.e. microwells). The printer can print fine features with a width less than 200 μm in a positive manner.

FIG. 64 illustrates direct generation of high-aspect-ratio and multi-height structures using 3D printing.

FIG. 65 is a workflow of the double replica procedure when fabricating an example of the disclosed modular chip. Multiple PDMS molds carrying high-resolution negative patterns can be generated from one 3D-printed positive mold. After non-adhesive treatment, these negative molds can be used to generate positive channels to make the final device.

FIG. 66 illustrates quantitation of sorting performance when performing antibody-mediated negative selection and multimer-mediated positive selection to isolate HA-reactive T cells from peripheral blood cells using an example of the disclosed system.

Similar reference numerals may have been used in different figures to denote similar components.

### DESCRIPTION OF EXAMPLE EMBODIMENTS

In various examples, the present disclosure describes devices and methods for recovery of target cells from a collection of cells, in particular magnetically tagged target cells. The disclosed devices, systems, and methods may also be used to recover target cells from fluid sources (carrying a collection of cells) such as peripheral blood, vascularized tumors, malignant pleural effusion, lymphatic fluid, the fluid portion of bone marrow, and other cell-carrying fluids. In one example, the method is a tunable immunomagnetic cell sorting approach that may be used to enable rapid and efficient recovery of TILs from solid tumors.

Referred to as microfluidic targeting of infiltrating cells (or MAGIC), it may be used for the recovery and expansion of TILs from tumor tissues based on immunomagnetic sorting. Although the present disclosure provides examples where cellular recovery is performed on TILs from a tumor sample, the disclosed methods and devices may be suitable for magnetic profiling of other cells in various mediums, with modification as appropriate. In another example, the disclosed devices, systems, and methods may be used to collect tumor-reactive immune cells from peripheral blood.

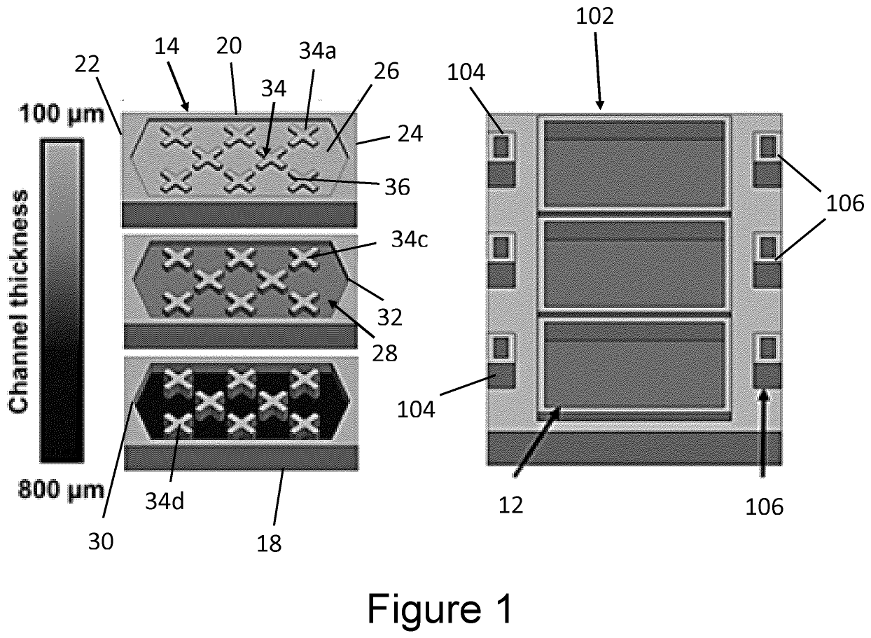

MAGIC uses a series of modular microfluidic chips that are designed for configurable, quantitative, and volumetric cell separation. Referring to FIGS. 1-6, there is shown devices 10 and systems 100 for recovering of magnetically tagged target cells from a fluid collection of cells according to example embodiments.

Device 10 generally comprises a magnet plate 12 and a modular chip 14 that is releasably coupled to, and overlies, magnet plate 12.

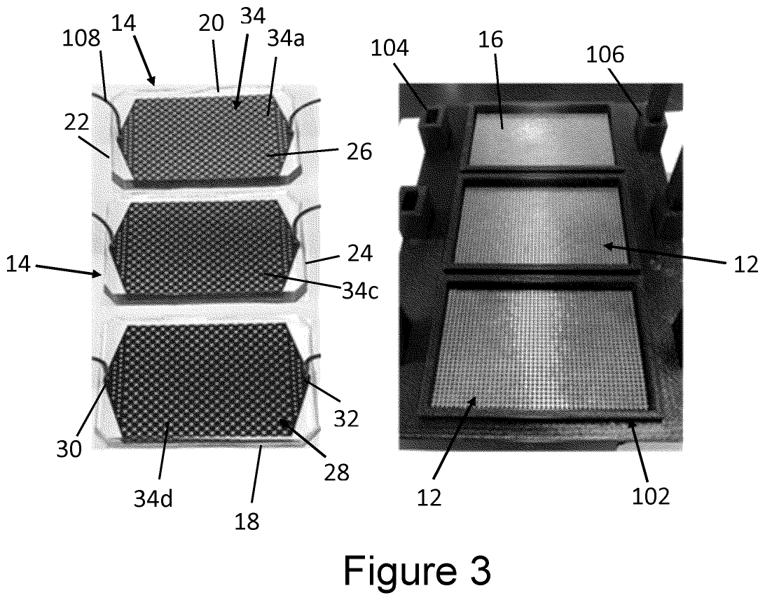

As best seen in FIG. 3, each magnet plate 12 has an array of micro-magnets 16 positioned and arranged therein, where the array of micro-magnet 16 produces a generally constant magnetic field along magnet plate 12. In the shown embodiment, all micro-magnet 16 are N52 NdFeB magnets. In other applications, magnets having differing magnetic strengths may be used. In yet further applications, magnets having different magnetic strengths may be secured to the same magnet plate 12 to create a varying magnetic field along magnet plate 12.

Modular chip 14 is made up of a base and chamber walls 20. In the present embodiments, modular chip 14 has a first end 22 and an opposed second end 24. The base may be a glass base 18 with a flow side 26 and an opposed coupling side for coupling with magnet plate 12. Flow side 26 and the opposed coupling side generally extend between first and second ends 22, 24. At least flow side 26 of glass base 18 may also be coated with polydimethylsiloxane (PDMS) or another suitable coating to prevent non-specific capture by smoothing the surface, such as another silicone based lubricant. This coating helps to make flow side 26 of glass base 18 smooth, so captures cells can slide off during recovery. The coating also helps to form a bond between chamber walls 20 and glass base 18 to prevent leakage.

Glass base 18 may gave a thickness between 0.05 mm and 0.5 mm. Preferably, glass base 18 has a thickness of no more than 0.1 mm, so as to better allow the magnetic field exerted by micro-magnets 16 to extend past glass base 18 when magnet plate 12 and modular chip 14 are coupled together. The dimensions of glass base 18 may be 30 mm to 300 mm long, and/or 12.5 mm to 125 mm wide. In the present embodiments, glass base 18 is 75 mm long and 50 mm wide.

Chamber walls 20 extend from flow side 26 of glass base 18 and may be secured thereto, such as by using an adhesive. Chamber walls 20 and glass base 18 collectively form a flow chamber 28 therebetween. Flow chamber 28 has an inlet 30 positioned at one end of flow chamber 24, such as proximate first end 22 of modular chip 14. Flow chamber 28 also has an outlet 32 positioned at an opposed end of flow chamber 24, such as proximate second end 24. In the shown embodiments, each device 10 has one inlet 30, one outlet 32, and one flow chamber 24. However, in alternate applications, device 10 may have multiple inlets 30, and/or multiple outlets 32, and/or multiple flow chambers 28.

While inlet 30 is configured to receive a fluid into flow chamber 24, and outlet 32 is configured to discharge the fluid out of flow chamber 24, inlet 30 and outlet 32 may be used in reverse. In such a case, inlet 30 becomes outlet 32, and outlet 32 becomes inlet 30.

Modular chip 14 further includes a plurality of flow rate-reducing structures 34 positioned within flow chamber 28. Flow rate-reducing structures 34 also extend from flow side 26 of glass base 18 and may be secured thereto. In some examples, chamber walls 20 and flow rate-reducing structures 34 may be formed as a single unit, such as through 3D printing and injection molding, which is then secured to glass base 18. In alternate applications, chamber walls 20 and flow rate-reducing structures 34, or device 10 as a whole, may be formed through injection molding. Each structure 34 comprises a trapping surface 36 that is shaped to reduce flow rate in a vicinity of trapping surface 36. In the present embodiment, each flow rate-reducing structure 34 is X-shaped, though other shapes may be used, such as V-shaped or C-shaped geometry.

The magnetic field produced by the array of micro-magnets 16 extends through glass base 18 and cooperates with the flow rate-reducing structure 34 to define a respective capture zone in the vicinity of each of flow rate-reducing structures 34.

Depending on the desired rate/type of filtration, the height of each flow rate-reducing structure 34 may be between 50 microns and 800 microns. If the height is beyond 800 microns, it was found that the mechanism of cell capture changes, and specificity of capture was lost. If the height of flow rate-reducing structure 34 is lower than 50 microns, it was found that clogging resulted, since cell sizes are ˜20 micron.

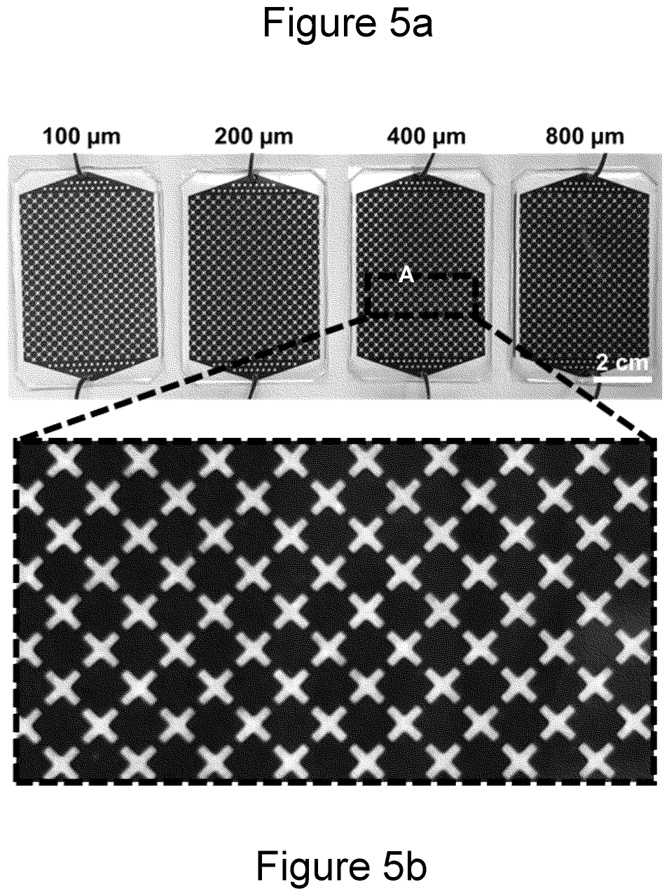

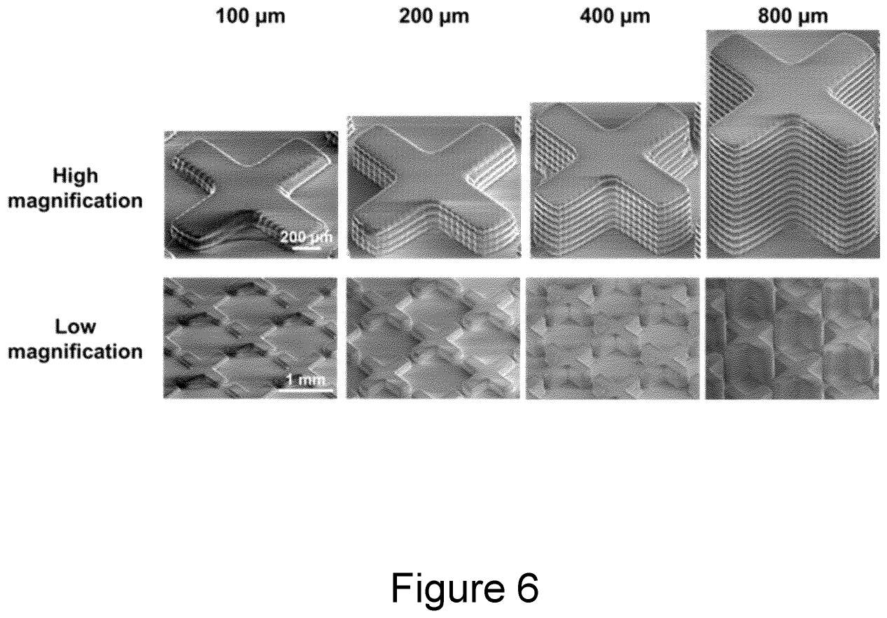

In the embodiments depicted in FIG. 1, for example, the height of all of the flow rate-reducing structures in the top left modular chip 14 is 100 μm (34a), the height of all of the flow rate-reducing structures in the middle left modular chip 14 is 400 μm (34c), and the height of all of the flow rate-reducing structures in the bottom left modular chip 14 is 800 μm (34d). In the embodiments depicted in FIGS. 5a, 5b, and 6, flow rate-reducing structures 34a, 34b, 34c, and 34d with heights of 100 μm, 200 μm, 400 μm, and 800 μm, respectively, are shown in further detail.

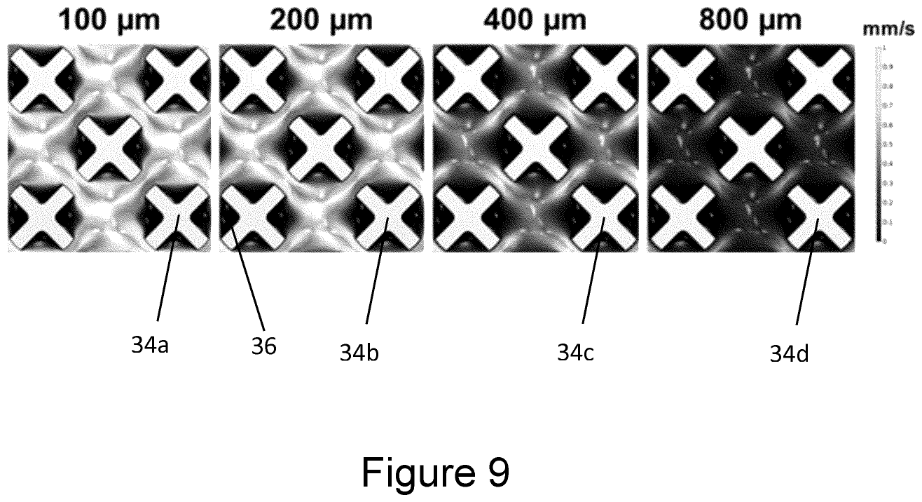

In further applications, modular chip 14 may have flow rate-reducing structures 34 with heights that differ from one another on the same glass base 18. For example, modular chip 14 shown in FIG. 9 includes flow rate-reducing structures 34a, 34b, 34c, and 34d arranged in increasing order in terms of height on glass base 18.

To help determine or select the appropriate height(s) of the flow rate-reducing structures for a given modular chip flow, cytometric profiles may be used to help design the sorting setup to capture certain targeted populations.

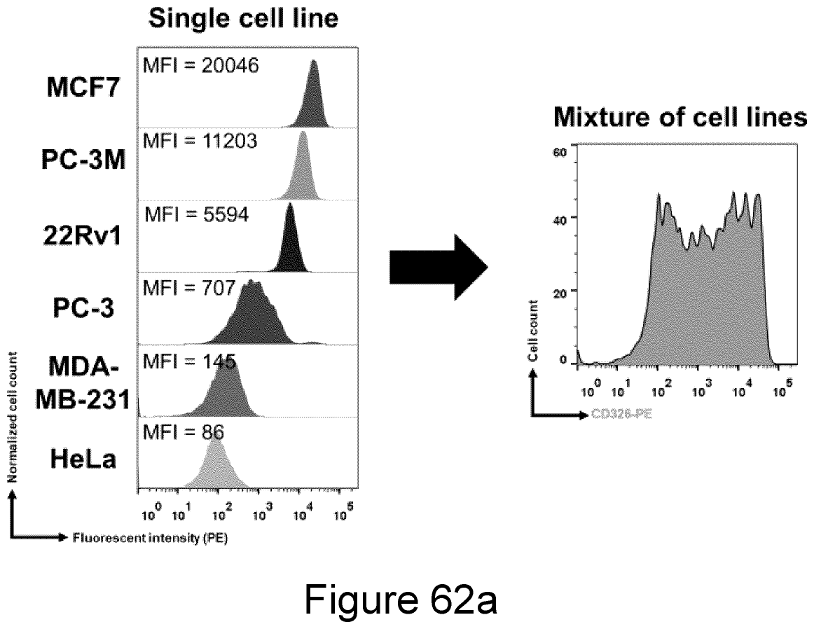

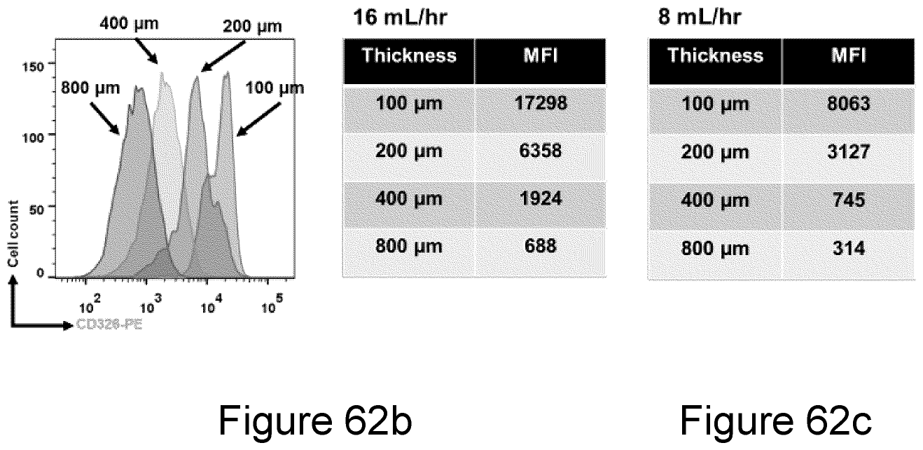

In one application, a series of human cancer cell lines that express CD326were benchmarked by flow cytometry, including MCF-7, PC-3M, 22Rv1, PC-3, MDA-MB231 and HeLa, as shown in FIG. 62a. These cells have a significant difference in CD326 expression, ranging from 80 to 20,000, as judged by the median fluorescence intensity (MFI). A quantitative sorting setup was configured that serially connected four modules, 100, 200, 400, and 800 μm, and the cell mixture was sorted under the flow rate of 16 mL/hr. FIG. 62b shows the MFIs of the population being captured at each module. Curve fitting suggested a linear relationship between log₂ thickness and log₂ MFI (R²>0.998) with a slope of around −1.5. Therefore, from the above observation, the MFI was to drop to ⅓ (2−1.5=0.35) when doubling the height of the capture module.

Then, based on the flow cytometric data of CD39 shown in FIG. 62b, the difference of the MFI between CD39^{high} and CD39^{low} populations was found to be about 27-fold (18615 vs 688). Hence, it was determined that the population would be well separated by 100 μm and 800 μm modules. The CD39^{med} population in-between was found to have a MFI of 5768, resulting in a fold change of 8.38 as compared to CD39^{low}. This provides a rationale to use a 200 μm module for capture. Taken together, a setup serially connecting 100, 200, and 800 μm modules may be selected for the quantitative sorting of CD39.

Of course, in alternative applications, modular chip 14 may have flow rate-reducing structures 34 with different heights than the ones shown and discussed herein.

By way of a further possible variation, the embodiment of device 10 shown in FIG. 9 includes two magnet plates 12 with modular chip 14 sandwiched in between. The magnets in this embodiment create a magnetic field with constant magnetization along all of flow rate-reducing structures 34a, 34b, 34c, and 34d.

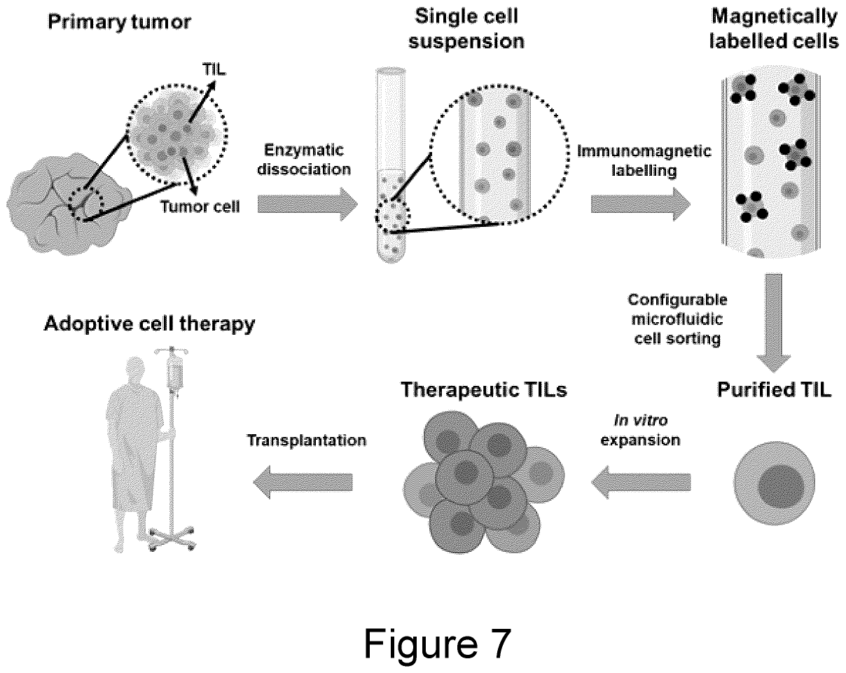

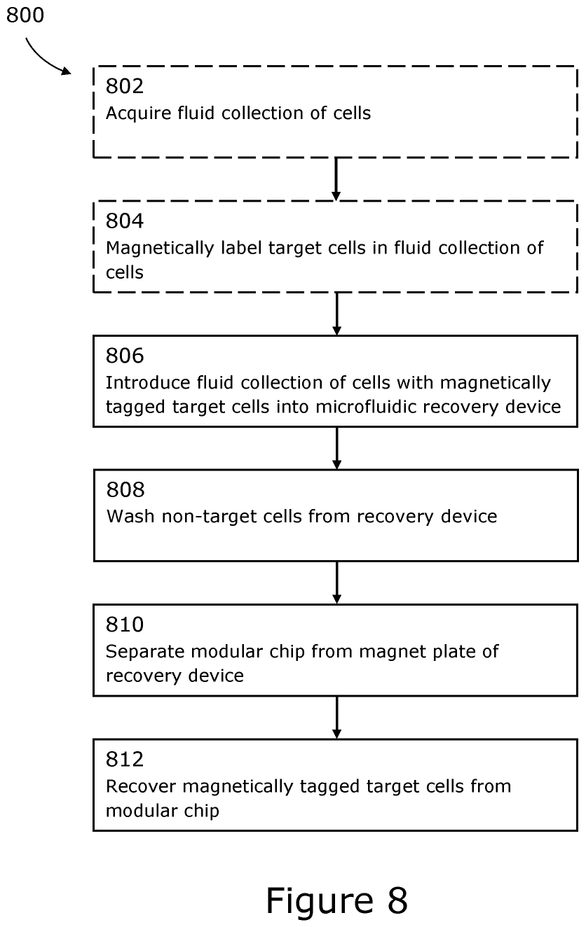

A method 800 for recovering magnetically tagged target cells from a fluid collection of cells using device 10 is shown in FIGS. 7 to 11 according to example embodiments.

At 802, method 800 may optionally include acquiring a fluid collection of cells. This acquiring may involve dissolving a tumour sample from a patient into single cells, forming the fluid collection of cells. The dissolving may further involve enzymatically dissociating the tumour sample into a single-cell suspension.

Alternatively or additionally, rather than dissolving a tumour sample to form the fluid collection of cells, the fluid collection of cells may be from peripheral blood, vascularized tumors, malignant pleural effusion, lymphatic fluid, the fluid portion of bone marrow, or other cell-carrying fluids holding single cells in suspension.

At 804, the single cells may then be labelled with magnetic particles, such as through immunomagnetic labelling. For example, the single cell mixture may be labelled by antibodies conjugated with magnetic nanoparticles (MNPs) that are specific to a surface marker expressed on immune cells of interest, but not on tumor cells (e.g. CD4, CD8, or CD45). With the attachment of MNPs on its surface, immune cells of interest (i.e. TILs) would obtain a strong magnetization within the microfluidic device. The magnetically tagged target cells are thus made to be susceptible to a magnetic attraction force.

The fluid collection of cells containing the magnetically tagged or labelled target cells are then introduced into device 10 at 806. In particular, the fluid collection of cells may be introduced into flow chamber 28 of device 10 through inlet 30 using a syringe (not shown).

When introduced into device 10, the fluid collection of cells experience two major forces, the magnetic force generated by the interaction between MNPs and the magnetic field generated by micro-magnets 16, and a fluidic drag force which is defined by the fluidic velocity in a specific region. When the magnetic force overcomes the fluidic drag force, a cell would acquire enough force to stay in a specific region. As the TILs are labeled with a higher number of MNPs, they experience a higher magnetic force compared to the tumor cells and other non-target cells.

Thus, the magnetic field from micro-magnets 16, in cooperation with the flow rate-reducing structure 34, in the capture zone is sufficiently high to overcome drag force on the target cells to promote capture of the magnetized target cells, from the collection of cells, in the capture zone. In that manner, the cells with higher magnetization are captured by flow rate-reducing structures 34.

As noted above, flow rate-reducing structures 34 may have different heights/thickness selected for different purposes. After the selection of a proper/desired configuration, flow rates may also be adjusted for better sorting performance. The differences in the thickness/height of modular chip 14 may itself contribute to different flow rates. Devices 10 with lower flow rate-reducing structure 34 may tend to have a lower volume in their flow chamber 28, and therefore, a higher flow rate therethrough. Conversely, devices 10 with higher flow rate-reducing structure 34 may tend to have a higher volume in their flow chamber 28, and therefore, a lower flow rate therethrough.

The flow rate itself may also be adjusted when the fluid collection of cells are injected into the recovery device 10. Returning to the cancer cell lines setup described above, the MFI from each module sorted at the flow rate of 8 mL/hr (FIG. 62c) was compared with 16 mL/hr (FIG. 62b). Collectively, the MFI was nearly linearly proportional to the flow rate with the k≈1. This provided a direct conversion to achieve desired MFI by tuning the existing flow rate.

For the above described setup (with serially connected 100, 200, and 800 μm modules) for the quantitative sorting of CD39, running a preliminary sorting under the flow rate of 8 mL/hr for the weak markers (i.e. CD39/PD-1) or 32 mL/hr for the strong markers (i.e. CD45) is recommended. After sorting, populations from each module can be collected to determine the MFI by flow cytometry. By comparing the existing MFI with desired MFI, a user would be able to extrapolate the optimal flow rate. Alternatively, the MFI by flow cytometry may be deduced by counting the percentage of cells in each module, followed by mapping these percentages in the flow cytometry data obtained above to infer corresponding MFI.

For example, CD39 was sorted with the flow rate of 8 mL/hr and the MFI from the 200 μm module was found to be 7620. The ideal MFI is 5768 (see cancer cell lines setup above). Therefore, the optimal flow rate in that case may be 8×5768÷7620=6.05 mL/hr.

The difference in flow rate allows for capture of cells with different expression of protein markers (i.e. cells with different degrees of magnetization). A specific amount of MNP is conjugated on an antibody targeting CD39. The CD39 antibodies bind to the CD39 protein specifically. Therefore, high expression of CD39 on cell surfaces yield to higher number of bound CD39 antibodies, which is proportional to the amount of MNPs. In this way, the expression level of CD39 correlates to the level of magnetic labelling. Thus, modular chip 14 with a lower thickness (and thus a higher flow rate), captures cells with higher expression. Modular chip 14 with a higher thickness (and thus a lower flow rate), captures cells with lower expression.

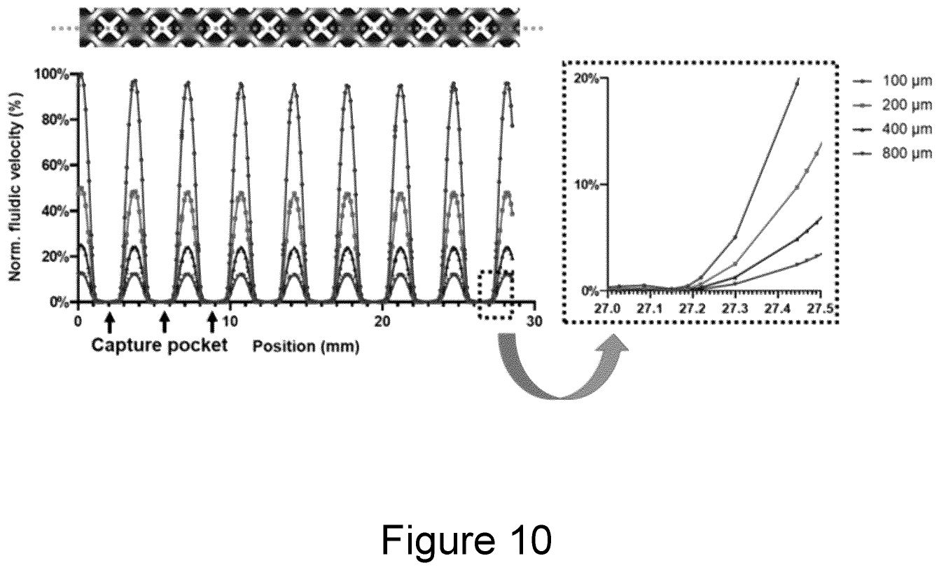

FIG. 9 shows a simulated flow velocity distribution within devices 10 with flow rate-reducing structures 34 of different heights, indicated by the unit of color bar (mm·s⁻¹). FIG. 10 shows quantitation of simulated flow velocity. The flow velocity near the pocket of the ‘X’-shaped structures 34 was low, forming a low velocity zone that favors cell capture, i.e. the capture zone.

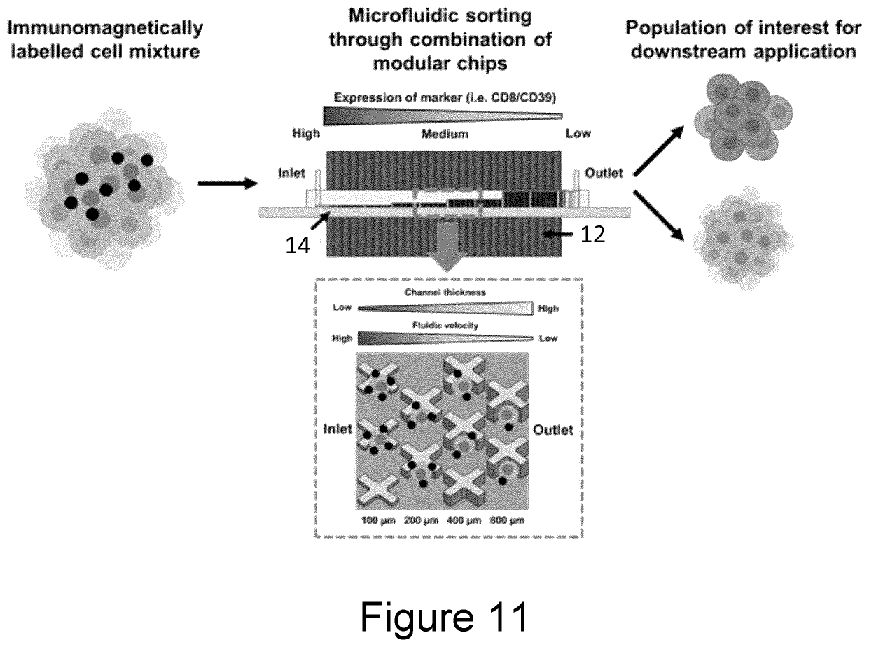

FIG. 11 is a figural illustration of how the shown embodiment of device 10 may capture magnetically tagged target cells for recovery. When immunomagnetically labelled cells are introduced into this device 10, the regions with differing heights between 100-800 μm, result in decreasing fluidic velocity and decreasing fluidic drag force. Specific cells are captured in the capture pockets of ‘X’-structure when the magnetic force it obtained overcomes the fluidic drag force.

At 808, method 800 further includes washing non-target cells out of device 10. The washing may be performed by introducing a flushing fluid into device 10 to wash out the non-target cells, or cells that were not captured in the capture zone. The flushing fluid may be injected through inlet 30 into flow chamber 28 using a syringe.

At 810, modular chip 14 is separated from magnet plate 12. The ability of modular chip 14 to separate from magnet plate 12 helps to improve throughput and increases the ease in removing cells from modular chip 14 after sorting. Separating modular chip 14 from magnet plate 12 removes the magnetic force generated by micro-magnets 16 from the magnetically labelled target cells. This allows for separation and recovery of the captured magnetically tagged target cells from modular chip 14 at 812.

In cases where the collection of cells are derived from a tumour sample, the recovered target cells may be CD8⁺ TILs. These recovered TILs may go through an in vitro expansion protocol with the stimuli of CD3/CD28 microparticles in an antigen-independent fashion. When expanded TILs reach desired levels, they may be transplanted back to in vivo environment (i.e. a patient) for adoptive cell therapy.

FIGS. 1-6 also illustrates systems 100 for sorting and recovering of magnetically tagged target cells from a fluid collection of cells according to example embodiments. System 100 generally comprises a first device 10a as described above, a second device 10b as described above, and a scaffold 102 that is configured to retain magnet plates 12 of first and second devices 10a, 10b.

As depicted, magnet plate 12 of each of first and second devices 10a, 10b is integrated into scaffold 102, positioned parallel to one another. Alternatively, magnet plate 12 of each of first and second devices 10a, 10b may be releasably securable to the scaffold. In that regard, magnet plates 12 may be coupled to scaffold 102 in any manner known in the art, such as via a sliding or snap-fit coupling mechanism. Magnet plates 12 having magnets of differing magnetic strengths or configurations may, thus, be interchangeably integrated into scaffold 102. In other alternate examples, first and second devices 10a, 10b may be arranged sequentially or at a non-perpendicular angle relative to one another on scaffold 102.

When system 100 includes more than one device 10, the flow rate-reducing structures of each device may be the same or different from one another.

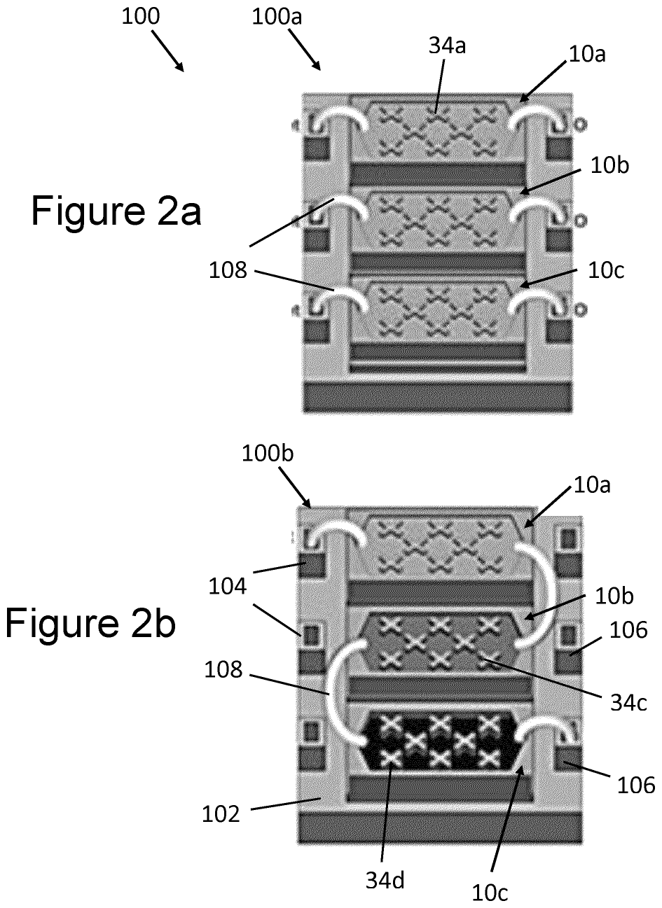

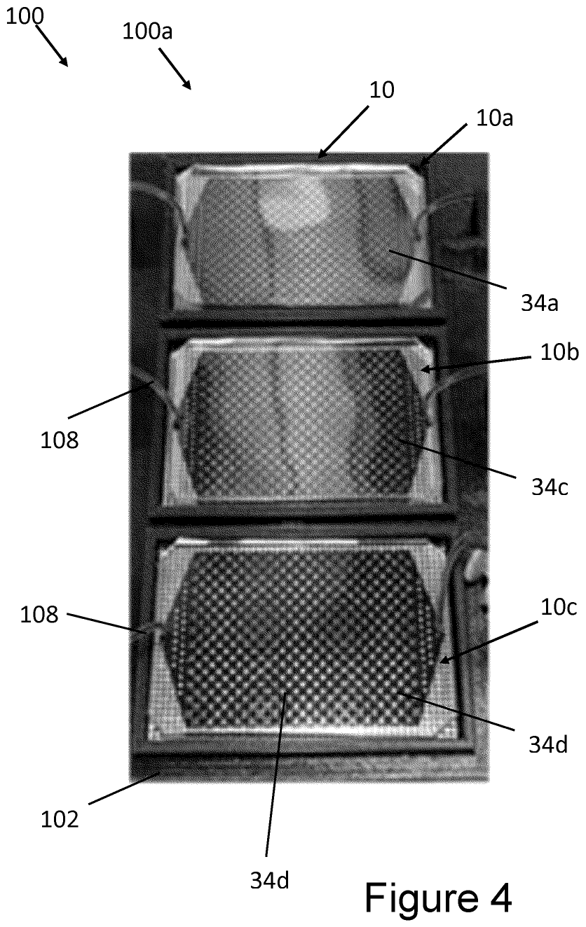

System 100 is further shown to include a first connector 104 positioned proximate an end of each of first and second devices 10a, 10b, and a second connector 106 positioned proximate another end of each of first and second devices 10a, 10b. For example, as shown in FIGS. 2a and 4, first connector 104 may be positioned proximate first end 22 of modular chip 14, while second connector 106 may be positioned proximate second end 24 of modular chip 14. Thus, first connectors 104 may be fluidly coupleable to the corresponding inlet 30 of each respective first or second device 10a, 10b, and second connectors 106 may be fluidly coupleable to the corresponding outlet 32 of each respective first or second device 10a, 10b.

The present embodiments use flexible tubing 108 to fluidly couple first connectors 104 with the corresponding inlets 30, second connectors 106 with the corresponding outlets 32, and/or an inlet 30 of one device with an outlet 32 of another device. The flexibility of tubing 108 allows it to be reconfigurable or reconnect-able between first connectors 104, second connectors 106 an inlet 30, and outlets 32. Alternate coupling mechanisms may be used instead of tubing 108 to fluidly couple first connectors 104 with corresponding inlets 30, and to fluidly couple second connectors 106 with corresponding outlets 32.

Each first connector 104 may also be in fluid connection with a source of the collection of cells (not shown), and each second connector 106 may also be in fluid connection with a residue container (not shown). FIGS. 1 to 4 further illustrate that scaffold 102 may be configured to hold more than two devices 10, such that additional devices (such as third device 10c), further to first and second device 10a, 10b, may be added to system 100 as desired.

The reconfigure-ability of the components of system 100 allows system 100 to be adapted into multiple modes or arrangements. Two example arrangements include a parallel configuration/system 100a, examples of which are shown in FIGS. 2a and 4, and a series configuration/system 100b, an example of which is shown in FIG. 2b.

In the embodiment of FIG. 2a, one example of parallel system 100a is shown where flexible tubing 108 fluidly couples each first connector 104 with inlet 30 of its corresponding device 10, another flexible tubing fluidly couples each second connector 106 with outlet 32 of its corresponding device 10. In such a case, each first connector 104 is also fluid connection with the source of the collection of cells, and each second connector 106 is also in fluid connection with the residue container (not shown).

As well, the height of flow rate-reducing structures 34 in each of first, second, and third devices 10a, 10b, 10c in FIG. 2a is the same. In particular, first, second, and third devices 10a, 10b, 10c include flow rate-reducing structures 34a, which have a height of 100 microns. In that manner, first, second, and third devices 10a, 10b, 10c of FIG. 2a are structurally the same. In alternate applications, the devices of FIG. 2a may instead include flow rate-reducing structures having a height between 50 microns to 800 microns.

In the embodiment of FIG. 4, another example of parallel system 100a is shown where the flexible tubing 108 is arranged in parallel, the same manner as that shown in FIG. 2a. However, the height of flow rate-reducing structures 34 in each of first, second, and third devices 10a, 10b, 10c is different. In particular, first, second, and third devices 10a, 10b, 10c of FIG. 4 include flow rate-reducing structures 34a, 34c, and 34d which have a height of 100, 400, and 800 microns, respectively.

FIG. 2b illustrates an example of series system 100b where first and second devices 10a, 10b, along with third device 10c, are arranged and fluidly connected in series.

In series system 100b, similar to parallel system 100a, connector 104 proximate first device 10a is fluidly coupled to inlet 30 of first device 10a with flexible tubing 108. However, as noted above, with each modular chip 14, the functions of inlet 30 and outlet 32 may be reversed. Thus, unlike parallel system 100a, outlet 32 of first device 10a is fluidly coupled to inlet 30 of second device 10b, not second connector 106. Moreover, outlet 32 of second device 10b is fluidly coupled to inlet 30 of third device 10c, not first connector 104. Outlet 32 of third device 10c is then fluidly coupled to second connector 106 proximate device 10c.

The height of plurality of flow rate-reducing structures 34 in first device 10a of series system 100b is also different (lower or higher) from the height of flow rate-reducing structures 34 in second device 10b and third device 10c. In the particular embodiment shown in FIG. 2b, first device 10a has flow rate-reducing structures 34a (100 μm), second device 10b has flow rate-reducing structures 34c (μm), and third device 10c has flow rate-reducing structures 34d (800 μm).

The modularity and configurability of system 100 allows for the system 100 to be field-programmable, i.e. allows for flexibility in changing the configuration for different sorting applications and to make the system 100 fit-for-purpose. For example, depending on the needs of the end user, the system 100 may be configured into parallel system 100a and can then be reconfigured into series system 100b, or a system having both parallel and series components and/or with a different combination of components.

In some examples, instead of the system 100 including first and second devices 10a, 10b each having its own magnet plate 12, the system 100 may include a single large magnet plate that can be used with first and second modular chips 14. The first modular chip 14 may be coupled to the scaffold such that the first modular chip 14 covers a first portion of the magnet plate 12, and the second modular chip 14 may also be coupled to the scaffold such that the second modular chip 14 covers a second portion of the magnet plate 12. Then the parallel system 100a or series system 100b may be similarly achieved by connecting the inlets 30 and outlets 32 of the first and second modular chips 14 as discussed above. That is, the modularity and configurability of the system 100 may be achieved using modular chips 14 that may be freely arranged on a single large magnet plate 12 (which may be integrated into the scaffold 102 or may be releasably coupled to the scaffold 102), or may be achieved using devices 100 that each includes a modular chip 14 with its own magnet plate 12.

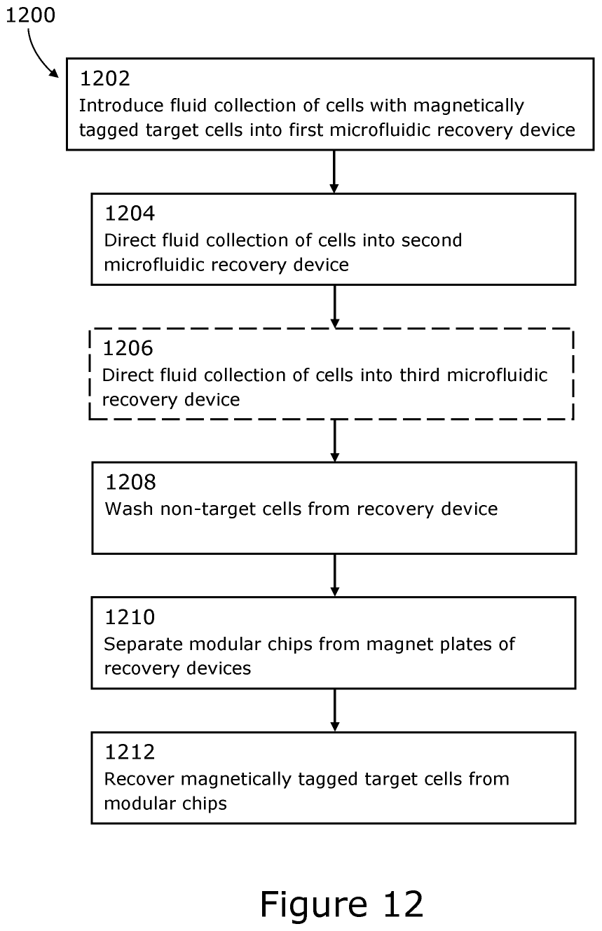

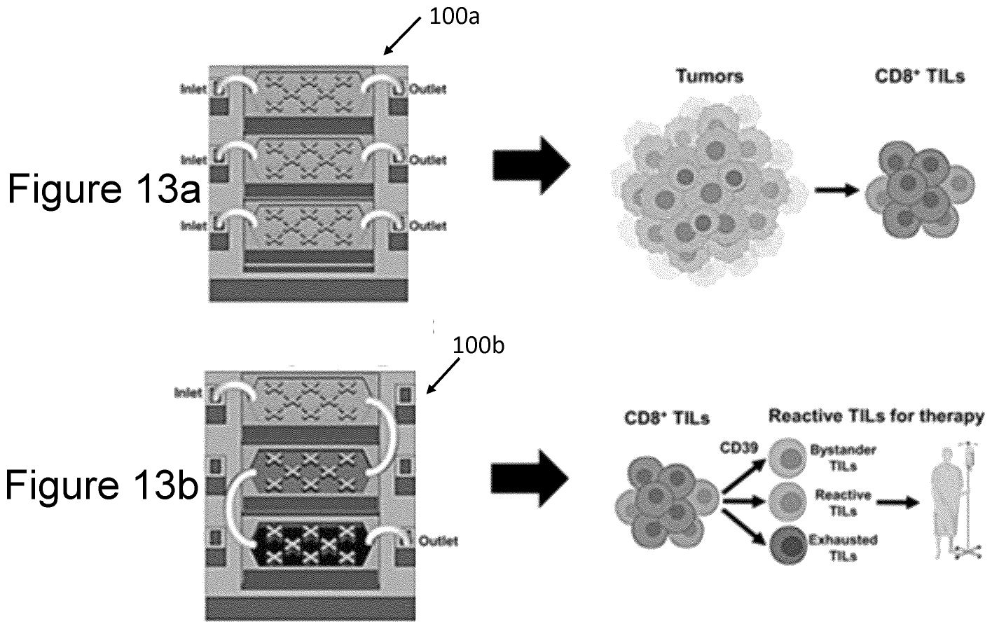

A method 1200 for recovering magnetically tagged target cells from a fluid collection of cells using system 100 is shown in FIGS. 12, 13a, and 13b according to example embodiments. Optionally, method 1200 may include the dissolving of the tumour sample (802) and the magnetic labelling of target cells (804) as discussed above.

At 1202, method 1200 then includes introducing the fluid collection of cells containing the magnetically tagged target cells into system 100, specifically into first device 10a through inlet 30 of first device 10a. At 1204, the fluid sample is then directed into second device 10b, i.e. from outlet 32 of first device 10a into inlet 30 of second device 10b. Method 1200 may also optionally include at 1206 directing the fluid sample into third device 10c, i.e. from outlet 32 of second device 10b into inlet 30 of third device 10c.

Non-target cells are then washed out of system 100 at 1208. The washing may be performed by introducing a flushing fluid into system 100 to wash out the non-target cells, or cells that were not captured in the capture zone. The flushing fluid may be injected through inlet 30 into flow chamber 28 of first device 10a using a syringe. The flushing fluid would then make its way through second device 10b and third device 10c as described above.

At 1210, modular chips 14 of first, second, and third devices 10a, 10b, 10c are separated from their corresponding magnet plates 12. Then at 1212, the magnetically tagged target cells from modular chips 14 of first and second devices 10a, 10b are recovered.

FIGS. 13a and 13b illustrate what method 1200 may be performed for when using system 100 in parallel and in series.

FIG. 13a includes parallel system 100a where first, second, and third devices 10a, 10b, and 10c are connected in parallel, and they each have flow rate-reducing structures 34a of 100 microns. Given their structural sameness, method 1200 may be performed for ultra-high throughput binary sorting of tumour cells into CD8+ TILs and recovery thereof. Also referred to herein as “binary MAGIC”, this binary sorting allows for isolation of T-cell population to generate bulk TILs.

However, the TILs may be further sorted into sub-populations based on their degree of activation. In particular, sub-populations of cells with more potent phenotypes may be targeted by honing in on particular proteins in TILs.

To that end, FIG. 13b shows series system 100b where first, second, and third devices 10a, 10b, and 10c are connected in series with differing flow rate-reducing structures 34a, 34c, and 34d (of 100, 400, and 800 microns, respectively). Given their structural differences, method 1200 may be performed for high throughput quantitative sorting and recovery of tumour cells (or general CD8⁺ TILs) into subpopulations based on CD39 expression, such as Bystander TILs, Reactive TILs, and Exhausted TILs. This type of sorting is also referred to herein as “quantitative MAGIC”.

As noted above, flow rate-reducing structures 34 with different heights can result in different flow rates through flow chambers 28. The labeled fluid collection of cells may also be injected at different flow rates. This difference in flow rate allows for capture of cells with different expression of CD39 protein markers. Thus, first device 10a with 100 micron flow rate-reducing structures 34a may captures cells with higher expression, such as Exhausted TILs. Second device 10b with 400 micron flow rate-reducing structures 34c may captures cells with medium expression, such as Reactive TILs. Third device 10c with 800 micron flow rate-reducing structures 34d may captures cells with lower expression, such as Bystander TILs.

Thus, when modular chips 14 of first, second, and third devices 10a, 10b, 10c are separated from their corresponding magnet plates 12, the target cells recovered from the first modular plate would largely be Exhausted TILs, the target cells recovered from the second modular plate would largely be Reactive TILs, and the target cells recovered from the third modular plate would largely be Bystander TILs.

Method 1200 may be performed following performance of method 800, where the magnetically tagged target cells recovered from method 800 are introduced into series system 100b. Method 1200 may alternately be performed independently from method 800, where the fluid collection of cells (such as the dissolved tumour sample) with magnetically tagged target cells are directly introduced into series system 100b.

The separability of modular chip 14 from magnet plate 12 in device 10, and the modular configuration of system 100 helps to increase the ease when removing cells from the modular chip, and allows for tunable resolution. Cells of a certain sub-population may be easily recovered, as any one compartment containing the cells of interest can simply be taken out separately from the other modules. This modular design also allows the end-users to assemble a sorting system that meets their demand in terms of resolution (number of sorted populations), throughput, and system complexity.

Another benefit of using system 100a in series is that it may achieve up to 30-fold higher recovery efficiency and 100-fold better throughput compared to commercial sorting technologies without sacrificing purity. High recovery and purity may be achieved that improve the initial quantity and diversity of clonotypes of TILs and accelerates the expansion process. TILs isolated using this approach need minimal expansion, which maximize their in vivo cytotoxic phenotypes. Using in vivo adoptive cell therapy in a murine tumor model, it was demonstrated that the TILs isolated and expanded through MAGIC platform were highly potent and could extend median survival of xenografted animals by 50%.

In addition, a quantitative sorting setup (system 100a in series) for the high-throughput, and fine profiling of TIL subpopulations based on CD39 (system 100b in parallel) may be achieved. It is demonstrated in the following examples that moderate levels of expression of CD39 defines a progenitor population of TILs that is antigen-specific, self-renewable, and able to rapidly differentiate into highly cytotoxic phenotypes. The characteristics of the CD39^{med} population yield excellent anti-tumor effects in vivo compared to CD39^{high}, CD39^{low} or bulk TILs. Taken together, the examples described herein demonstrate that the disclosed devices, systems, and methods enable cost-effective and efficient adoptive cell therapy with rapid turnaround.

### Example Fabrication Method

An example fabrication method of an example of the disclosed device is now described. The separability of the modular chip and the magnet plate, as separate components, allow for at least the modular chip to be fabricated more quickly and cheaply using 3D printing and/or injection modeling, rather than using conventional lithography. Further, by enabling a low cost, high volume fabrication process for the modular chip (e.g., using 3D printing), the modular chip may be a disposable part of the system. Since the modular chip is the component that is most in direct contact with the fluid collection of cells (which may be a biological fluid, such as blood, lymphatic fluid, etc.), the disposability of the modular chip may be beneficial for hygienic reasons and/or for use in a healthcare setting.

In this example, the mold for fabricating MAGIC/modular chip 14 was 3D printed by a stereolithographic 3D printer (Microfluidics Edition 3D Printer, Creative CADworks, Toronto, Canada) using the “CCW master mold for PDMS” resin (Resinworks 3D, Toronto, Canada) with the layer thickness of 25 μm. Other known 3D printing resins may be used in this process, optionally with a UV or thermal treatment.⁴⁹ The MAGIC chip was made by casting PDMS (Sylgard 184, 182 or 186 Dow Chemical, Midland, MI) on printed molds, followed by 30 min-4-hour incubation at 50-100° C. Cured PDMS replicas were peeled off, punched and plasma bonded to thickness no. 1 glass coverslips (260462, Ted Pella, Redding, CA) to finish the chip. Before use, the MAGIC chip was treated by 0.01-1% Pluronic F68 (24040032, Thermo Fisher Scientific, Waltham, MA) in phosphate-buffered saline (Wisent Bio Products, Montreal, Canada) for 30 min-24 hr to reduce non-specific binding between cells and chips. During experiments, each device was sandwiched by arrayed N52 NdFeB magnets (D14-N52, K&J Magnetics, Pipersville, PA) and connected to a digital syringe pump (Fusion 100, Chemyx, Stafford, TX) for fluidic processing.

Fabricated chips were sputter-coated with 15 nm Au (Denton Desk II, Leica) for imaging under a field-emission scanning electron microscope (SU5000, Hitachi, Tokyo, Japan) using 5 kV accelerating voltage and high-vaccum mode. In other applications, the fabricated chips may not be sputter-coated when used as described above.

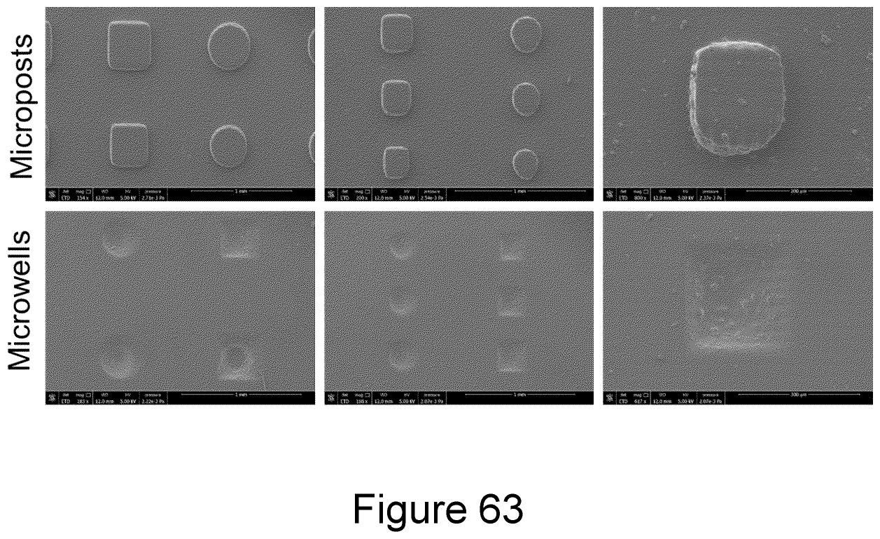

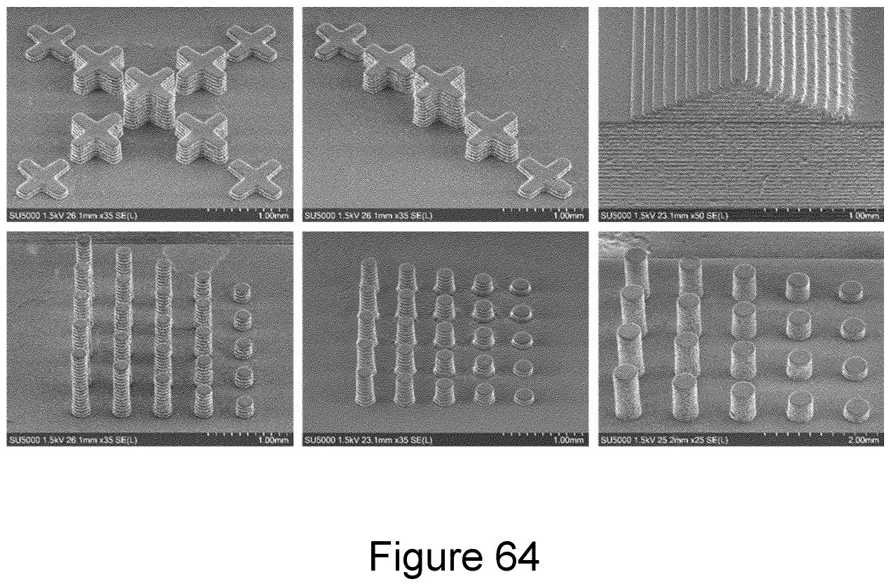

With regards to the 3D printing, the ability of a 3D printer to print positive (e.g. micropost) and negative (e.g. microwell) structures (see FIG. 63) was first quantified. It was found that the 3D printer in this example (i.e., Microfluidics Edition 3D Printer referenced above) has better resolution in printing positive structures compared to negative structures. The lower resolution of negative structures may come from the residue of resins staying within, due to surface tension or the high viscosity of resins. In that regard, the 3D printer may print high-resolution patterns, such as ‘X’-shaped cell capturing pocket, on positive structures. The 3D printer was found to support a fine structure with a height of at least up to 1 mm, which is seldom doable through standard lithography (see FIG. 64). In addition, the 3D printer was found to be able to generate multi-depth structures in one shot, while the standard lithography method requires multiple rounds, since lithography can only produce one thickness per round.

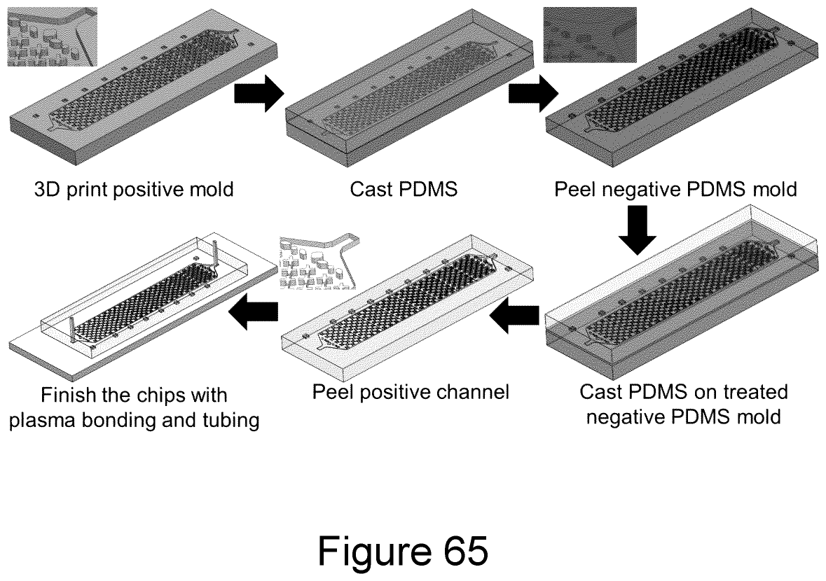

The challenge of limited resolution in 3D printing of negative structures may be addressed through the use of a specially designed double replica procedure of casting PDMS (see FIG. 65). This double replicate procedure firstly generates a PDMS mold carrying negative structures. The negative mold was then treated with the saturated detergent in 70% ethanol solution for 20 min-72 hours. This detergent treatment allows newly cured PDMS to be separated from the treated one, enabling the use of negative PDMS structure as a mold to generate positive channels during device fabrication.

The use of this protocol has at least three major advantages. Firstly, it allows any structure to be fabricated in a sufficiently high resolution. Since the 3D printer was found to have higher resolution printing positive structures, it is important to use this protocol to have the ability to make both positive and negative structures for channel fabrication. Secondly, this protocol can generate multiple negative molds from one 3D-printed piece—allowing the fabrication to be scaled up. At the same time, since all molds are formed from the exact same piece, it also minimizes the batch-to-batch variation during 3D printing/standard lithography. Thirdly, it is relatively cost-effective and straightforward as it does not involve the use of other types of resins and the treatment process is simple.

### Example Studies

The example device, fabricated as described above, was used in several example studies.

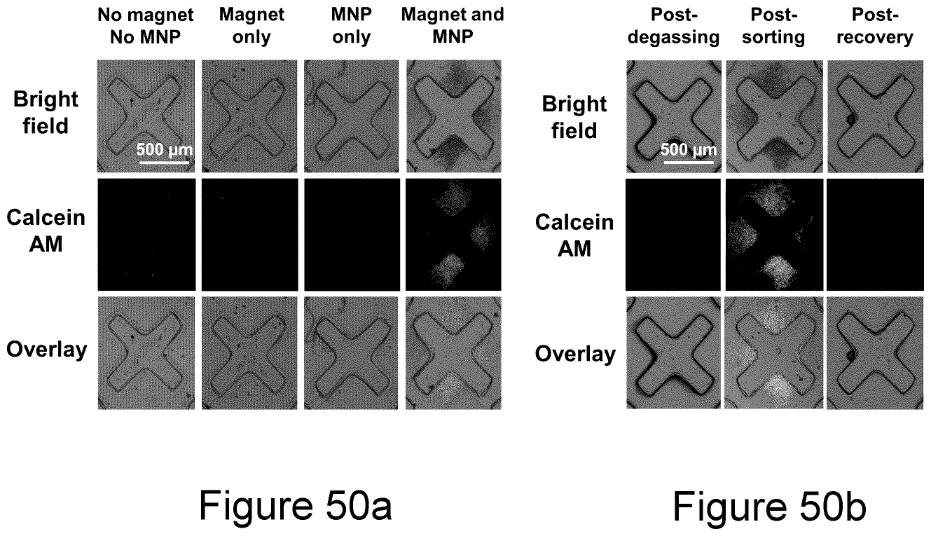

The overall design of the device was verified by sorting cells with/without MNPs and external magnets (FIG. 50a) using a binary sorting setup. As expected, cells were captured only when MNPs and external magnets were both present. It is noteworthy that the majority of cells were captured in the low-velocity pockets near ‘X’-shaped structures. This result proved the fidelity of our sorting principle and showed the specificity of our modular chips. In addition, the feasibility to recover captured cells from the chips was examined by flushing chips without external magnets (FIG. 50b). Nearly 100% of the captured cells were successfully removed from the chips, facilitating efficient cell recovery.

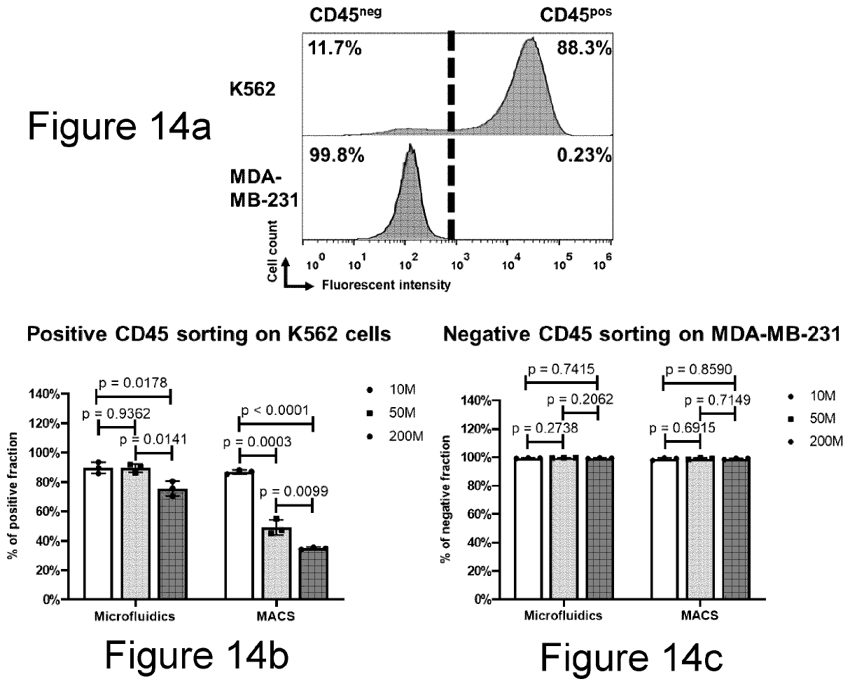

The sorting performance of MAGIC/device 10 and system 100 was compared with a commercialized magnetic sorting platforms—magnetic-activated cell sorting (MACS). For binary sorting with series system 100a, positive and negative sorting based on CD45 using K562 and MDA-MB-231 cells (see FIG. 14) was performed. During positive sorting, it was found that binary MAGIC offers consistent capture of cells up to 50 million per chip while MACS column saturated with 10 million cells and failed to capture the majority of positive cells at 50 million. This demonstrates that MAGIC is more suitable for applications involving volumetric capture of positive cells, such as purifying TILs from an excessive background of tumor cells.

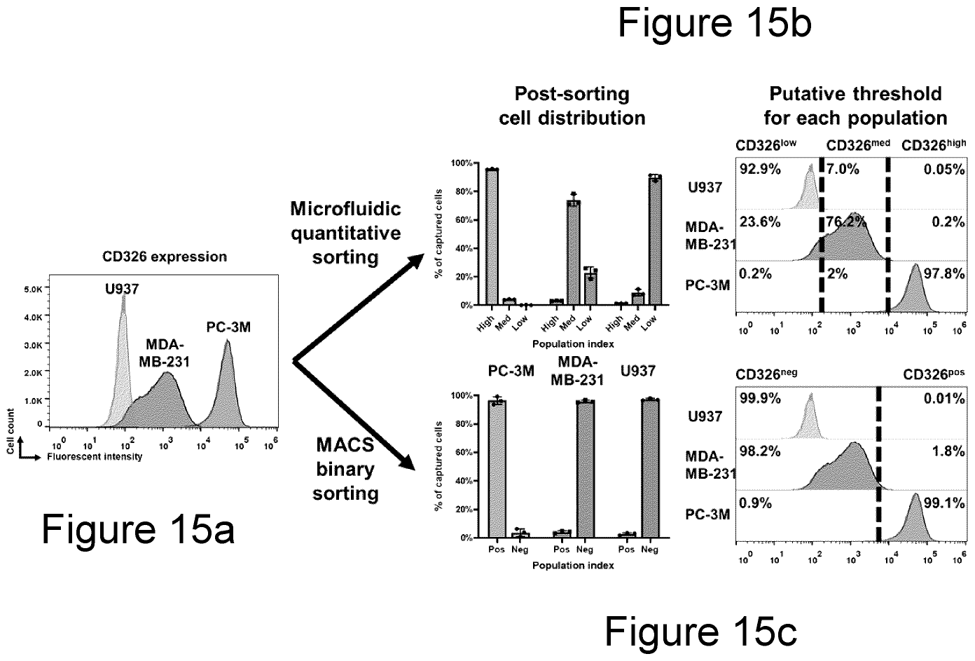

For the quantitative sorting with parallel system 100b, CD326 was tested using PC-3M, MDA-MB-231, and U937 cells that have high, medium, and low expression of CD326, respectively (see FIG. 15). It was observed that quantitative MAGIC could properly enrich all cell lines while MACS failed to capture CD326^{med} MDA-MB-231 cells in its positive fraction. This indicates MACS have limited ability to discriminate medium and low expressing cells. Quantitative MAGIC sorting offers a finer sorting resolution under a high throughput, which may better resolve biological information that is not accessible by MACS.

### Isolation of Cytotoxic CD8⁺ TILs Using Binary MAGIC

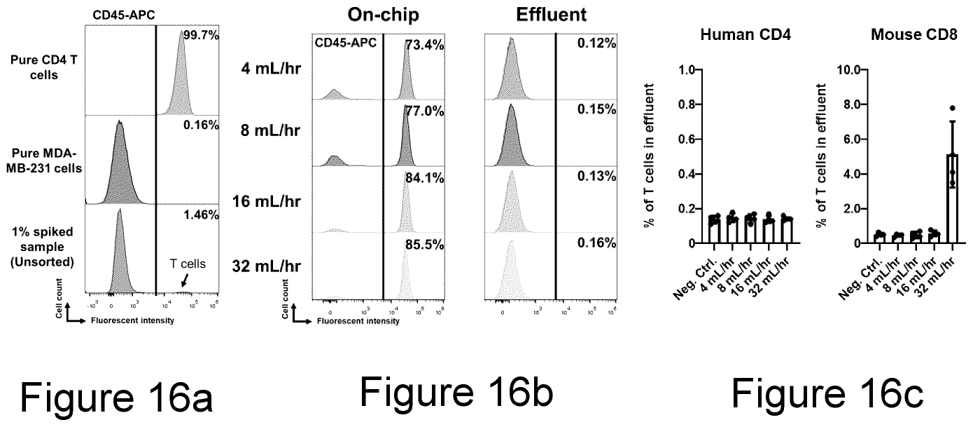

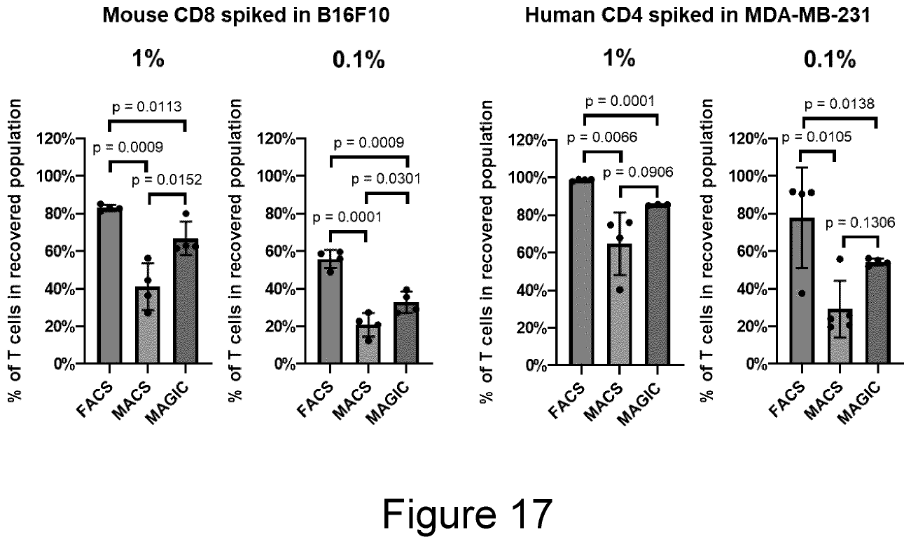

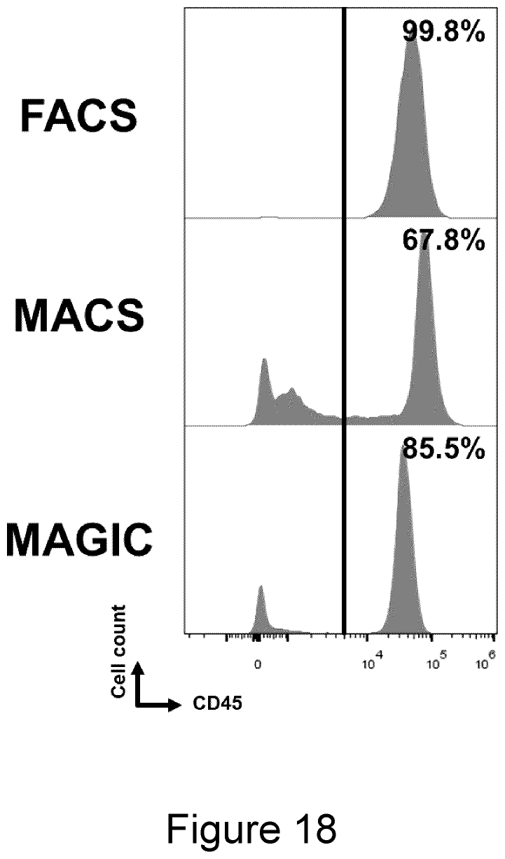

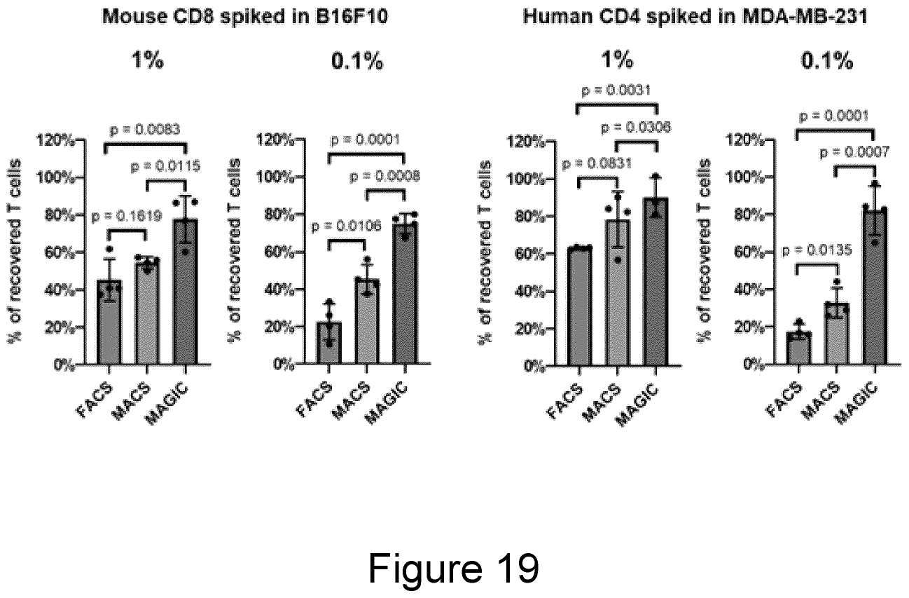

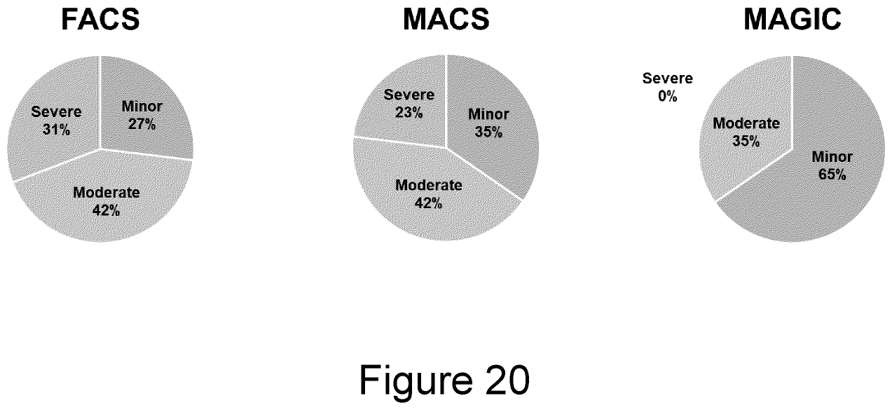

CD4 and CD8 are definitive markers for distinct anti-tumor T cell populations within tumors and are widely accepted for TIL isolation. The binary MAGIC setup (parallel system 100a) was configured for isolating TILs through CD4 or CD8. T cells were spiked in samples of tumor cells to optimize the flow velocity favoring the separation of TILs. Pure human CD4⁺ T cells and pure mouse CD8⁺ OT-1 T cells were used. The optimal flow rate for capture human CD4⁺ and mouse CD8⁺ T cells was found to be 32 mL/hr and 16 mL/hr, respectively (See FIGS. 16a to 17). This corresponds to a throughput of 320 million human cells and 160 million mouse cells per hour per device. As CD4⁺ and CD8⁺ TILs typically represent less than 10% of the total TILs,³⁷ such capacity should be enough for handling 500 million dissociated tumor cells per chip. The performance of MAGIC, FACS, and MACS was compared using small volumes of spike-in samples (˜10 million per run) from two distinct setups, CD4-mediated capture of human CD4⁺ T cells spiked in MDA-MB-231 human breast cancer cells, and CD8-mediate capture of mouse CD8⁺ OT-1 T cells spiked in B16F10 mouse melanoma cells (see FIGS. 18 and 19). It was found that MAGIC achieved consistent enrichment of T cells with the best recovery over FACS/MACS across stress to sorted cells compared to FACS/MACS, thanks to its droplet-free working principle and ultrahigh throughput (see FIG. 20).

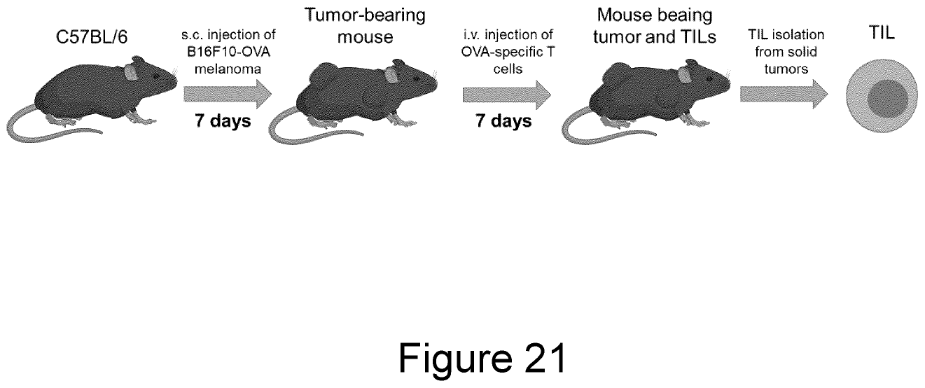

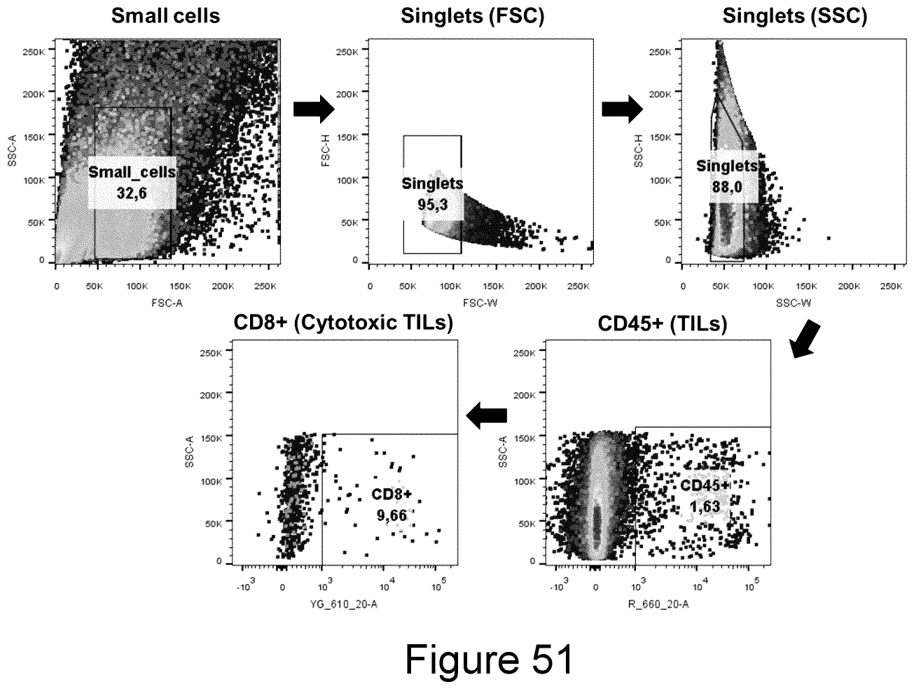

The MAGIC, FACS, and MACS were challenged with TILs from a B16F10 murine melanoma model (see FIG. 21). The mice were sacrificed on day 14 and TILs were isolated from the digested tumors using different isolation technologies based on CD8 expression. Interestingly, it was noticed that the percentage of CD8⁺ TILs only represents 0.2% of the total cell population within solid tumors (FIG. 51), and hence it is necessary to process all digested tumors to maximize the quantity of these rare populations. A single gram of tumor tissue typically contains 100 million-1 billion cells.³⁸ Such a high cell number makes the sorting of rare and live TILs from actual tumor samples very challenging. Processing undiluted samples with MACS or FACS was challenging due to clogging issues and required that samples were diluted to a concentration of 5×10⁶ cells/mL and 2×10⁶ cells/mL for MACS and FACS, respectively. This dramatically increased its processing time to 4 hours and 20 hours. MAGIC, however, was able to process concentrated samples with cell number up to 20×10⁶ cells/mL without clogging at a high flow rate per chip. The performance of MACS/FACS/MAGIC for sorting TILs from actual B16F10 tumors is summarized in Table 1.

TABLE 1

| | MAGIC | FACS | MACS |
|---|---|---|---|
| Subpopulations | 2-7, tunable | 2-4 | 2 |
| Purity¹ | 95.9 ± 2.4% | 75.9 ± 7.4% | 60.2 ± 8.1% |
| Recovery^{1,2} | 100.0 ± 13.6% | 2.5 ± 0.3% | 22.9 ± 3.4% |
| Enrichment ratio^{1,3} | 608.7 | 11.9 | 87.4 |
| Throughput per device | 320 × 10⁶ cells/hr | 2 × 10⁶ cells/hr | 50 × 10⁶ cells/hr |
| Throughput per setup⁴ | 3200 × 10⁶ cells/hr | 2 × 10⁶ cells/hr | 200 × 10⁶ cells/hr |
| Material cost | US$25 per setup | US$60 per hour | US$35 per column |
| Equipment cost | US$1,000-$2,000 | US$1,000,000 | US$2,880 |

1: B16F10 melanoma tumor as an example
2: Recovery normalized to the MAGIC group
$3 : Enrichment ratio = \frac{Purity after isolation}{Purity before isolation} \times Recovery rate$ 4: Each syringe pump contains 10 positions for setting up syringes for isolation. Each FACS machine contain 1 nozzle system for isolation. Each QuadroMACS contains 4 paralleled magnetic stands for isolation.

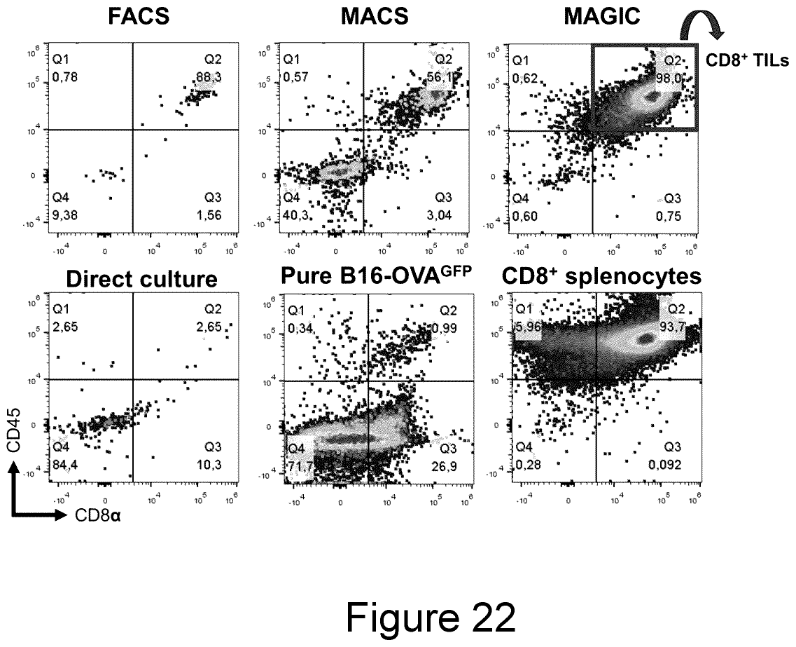

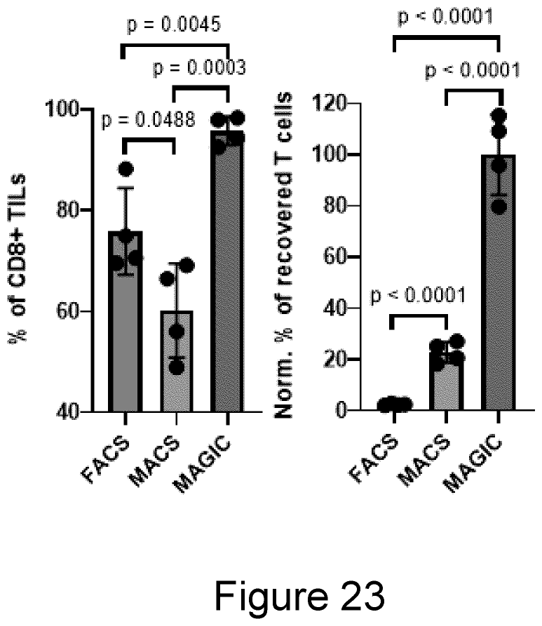

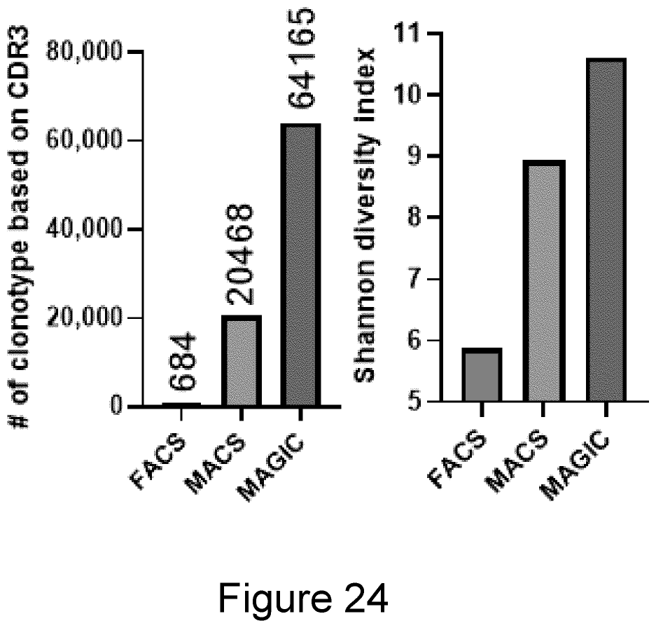

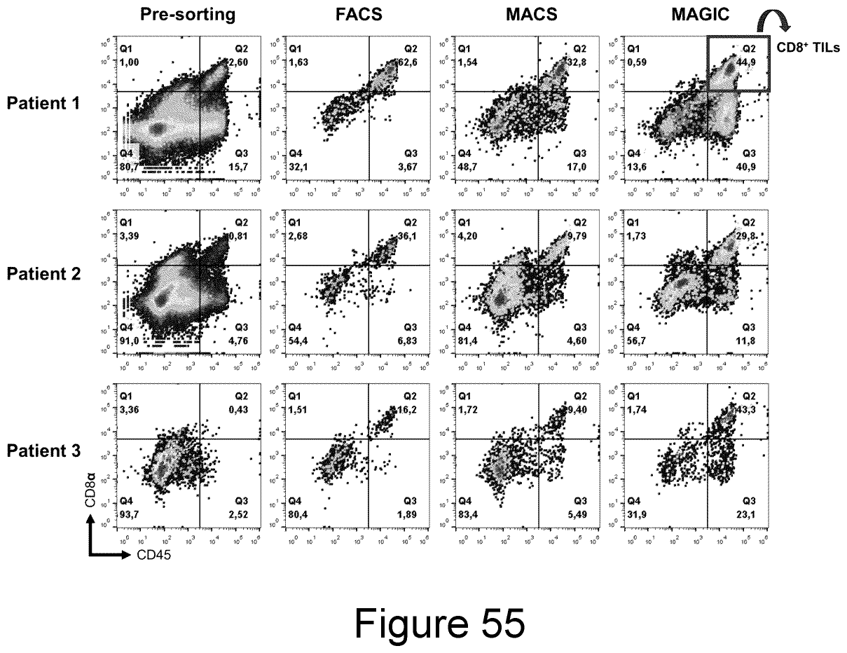

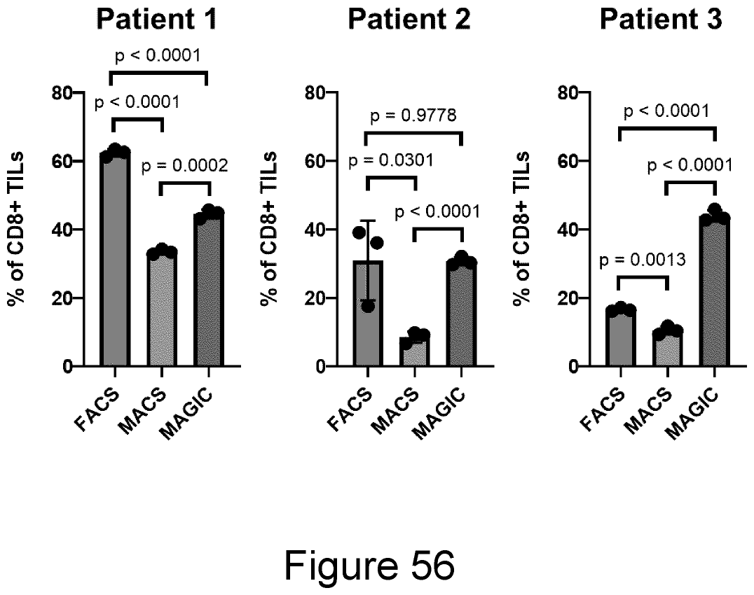

The purity and quantity of isolated CD8⁺ TILs were determined by CD8/CD45 co-staining after 4 days' culture under the medium formulated for T cell expansion (see FIG. 22), at which time over 97% of MNPs detached from the surface³⁴ to re-expose the CD8α epitopes for labelling. MAGIC achieved the best purity (98.0%) on day 4, and was more efficient than FACS (88.3%). This trend may be explained by the times required for FACS-based sorting as well as high levels of cellular stress that caused significant cell apoptosis/death. As shown in FIG. 23, MAGIC has 5-fold and 30-fold higher recovery than MACS and FACS, respectively. This trend is in good agreement with the spike-in characterization. A very limited number and percentage of TILs (2.65%) were recovered from unsorted digested tumor samples. This suggests that an efficient and gentle microfluidic sorting process leads to a better expansion curve of TILs. The TIL recovery was also tested using tumor specimens from non-small cell lung cancer (NSCLC) patients (see FIGS. 24, 55, and 56) and observed a similar trend—MAGIC retained the highest cell recovery and is 5 to 20-fold better than MACS and FACS.

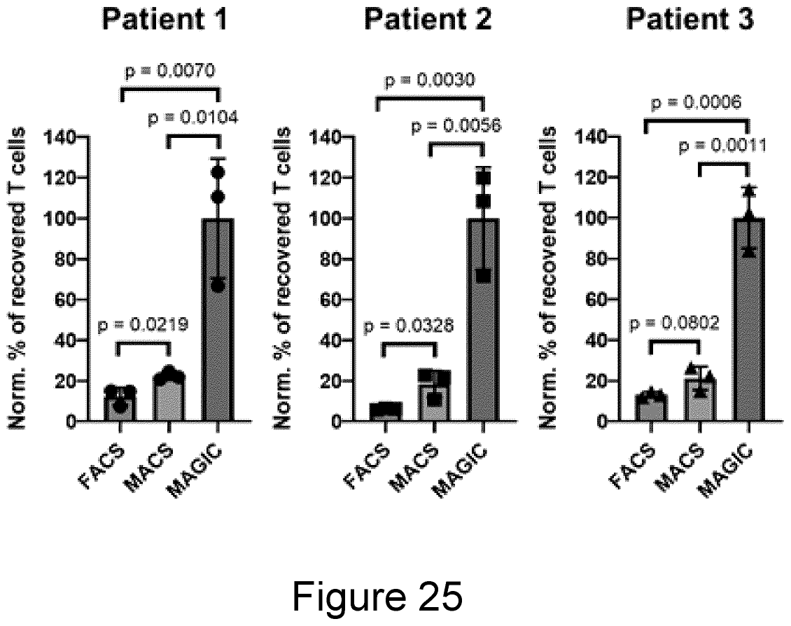

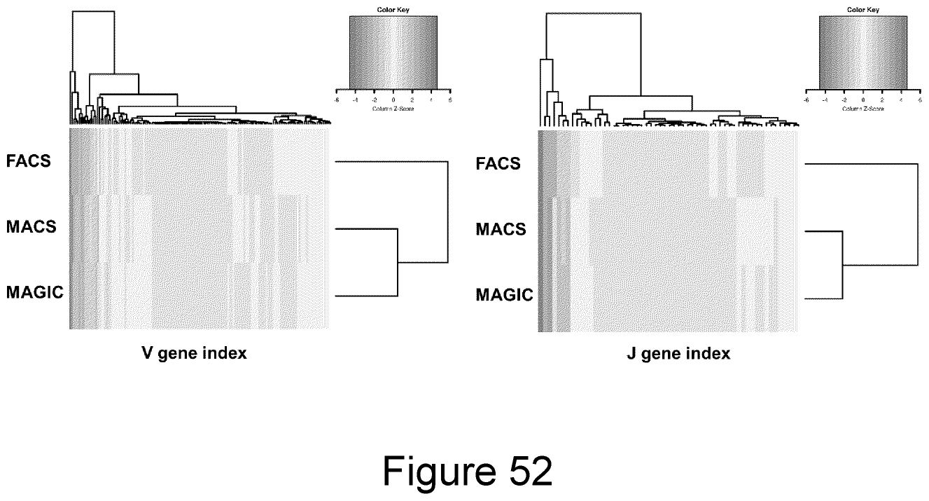

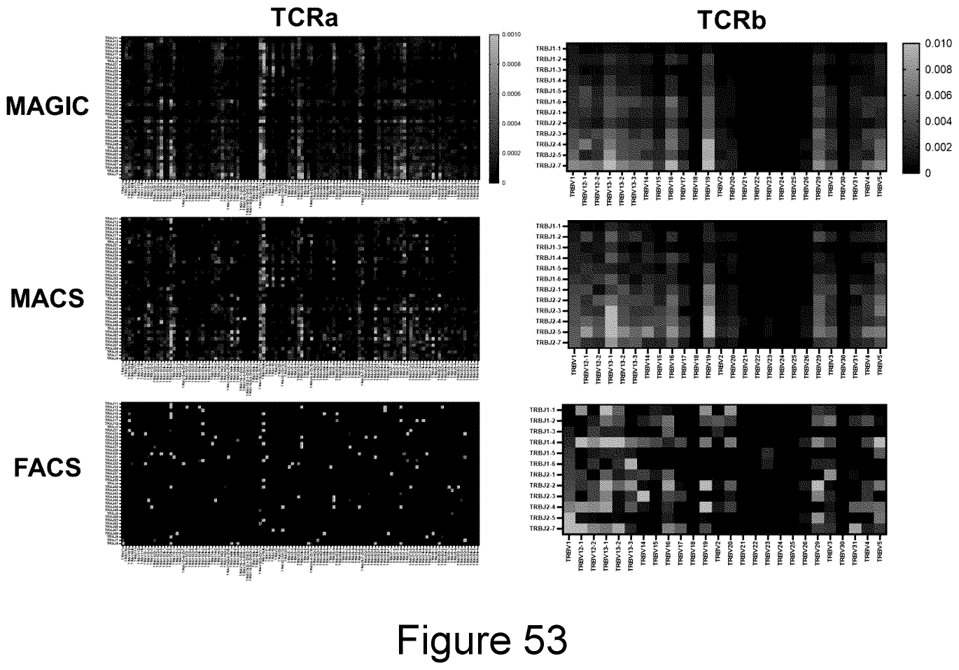

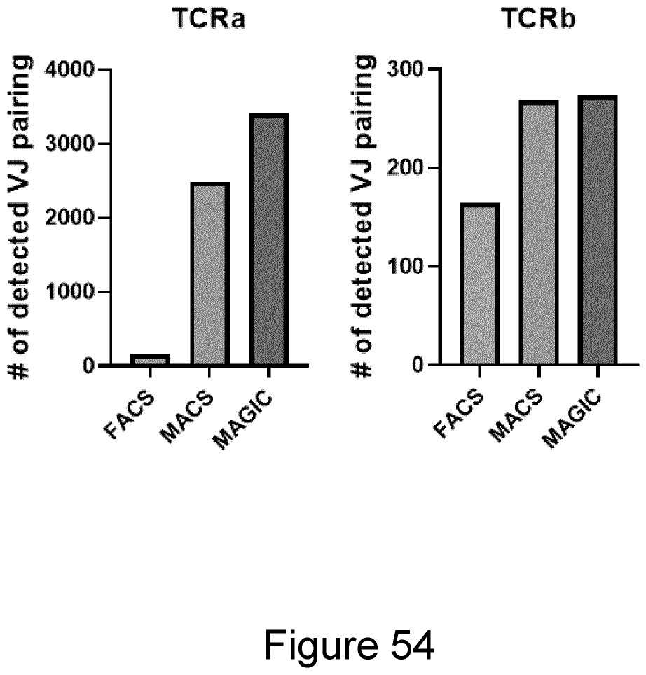

The clonotypes of the TILs isolated were analyzed using bulk TCR sequencing. The number of unique clones (based on CDR3) observed were 684, 20468, and 64165 for the TILs isolated by FACS, MACS, and MAGIC, respectively (FIG. 25). This trend suggests that the high recovery of MAGIC also helps to maximally preserve the heterogeneity and diversity of original TIL populations. Interestingly, different isolation technologies did not significantly alter the usage of V and J genes (FIG. 50), indicating a similar antigen specificity of isolated TILs. However, mapping of the V(D)J recombination (FIG. 51) reveals that the TILs isolated by MAGIC (referred to as MAGIC-TILs) have significantly higher complexity of V-J pairing and a higher Shannon diversity index (FIG. 52), which highlights the better diversity of TILs isolated by MAGIC.

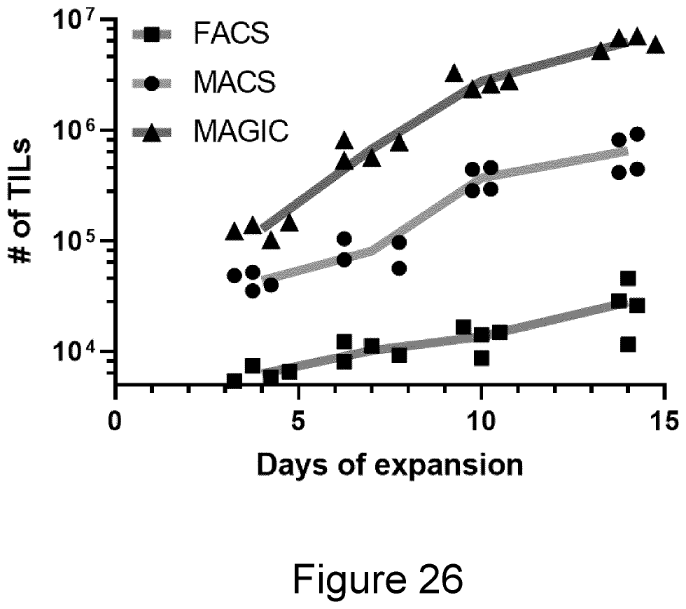

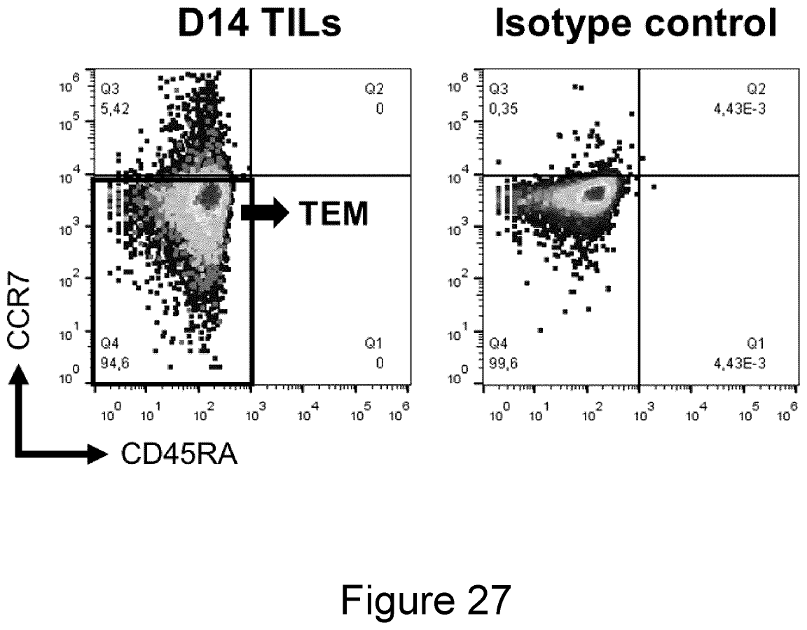

Having observed a significant improvement in quantity, purity, and diversity with the MAGIC isolation process, it was investigated whether enhancements in expansion rate and inherent cytotoxicity could also be observed. The TILs isolated was cultured using different methods for 14 days in well plates using a common CD3/CD28-based expansion protocol that subcultures cells at the density of 1×10⁶/mL. The cell number were recorded twice a week per well and calculated the total number of TILs isolated from each method (FIG. 26). The expansion rate of MAGIC-TILs was found to be faster than MACS/FACS-TILs. It takes 7 and 14 days for MAGIC and MACS TILs to reach a quantity of 1×10⁶ for each replicate. FACS-TILs was not able to supply a quantity of 1×10⁶ within 14 days due to its extremely low initial quantity, likely because cell density is a critical regulator for T cell activation and expansion.³⁹ The high yield provided by MAGIC results in more concentrated initial TIL populations that favor expansion. At the end of the expansion, the phenotypes of TILs by CCR7/CD45RA was characterized by staining (FIG. 27). About 95% of the TILs are CCR7⁻/CD45RA⁻, indicating a proper effector number of TILs favors the rapid in vitro expansion.

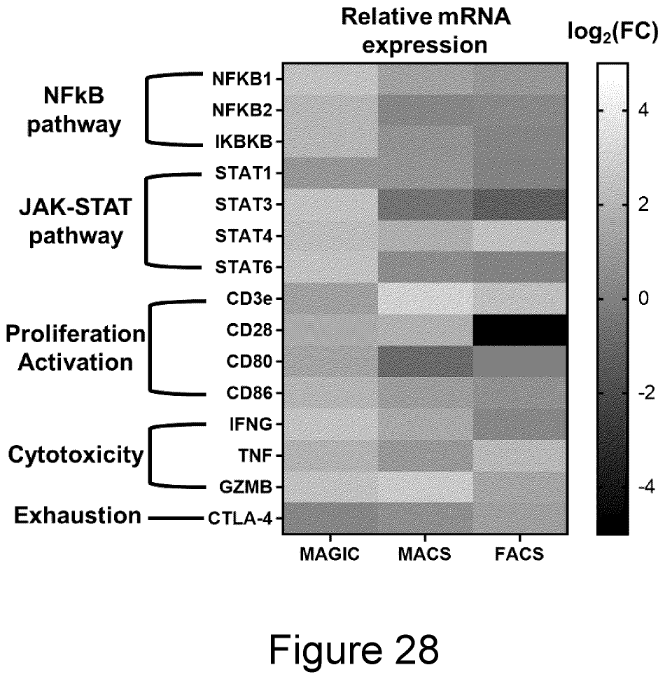

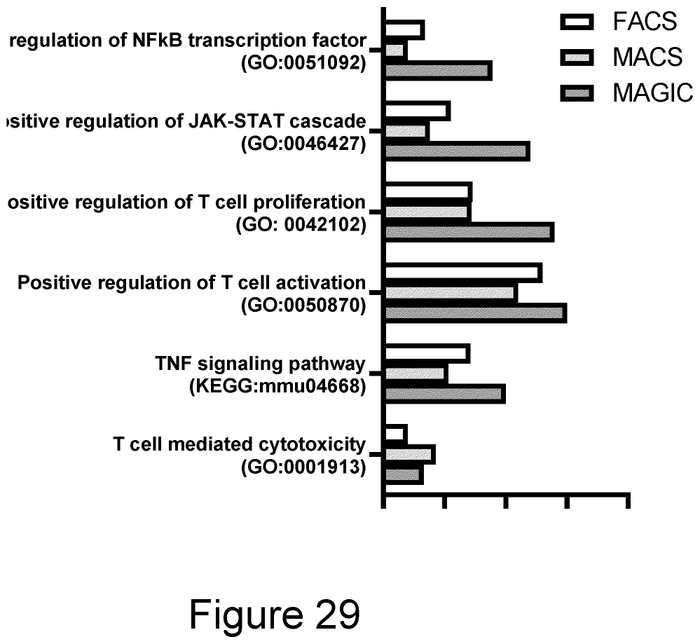

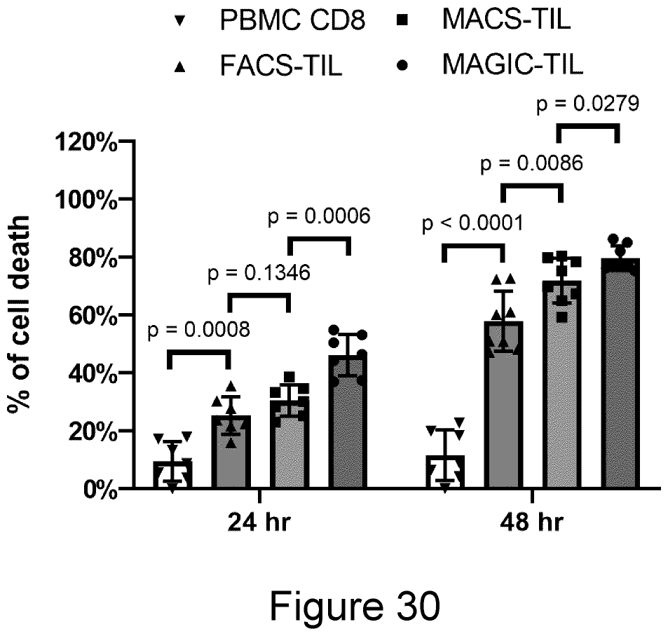

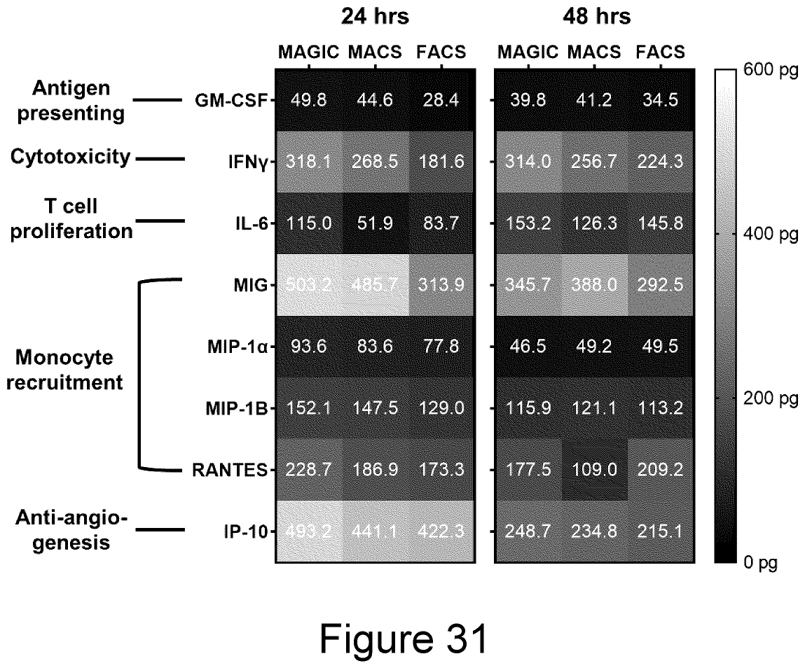

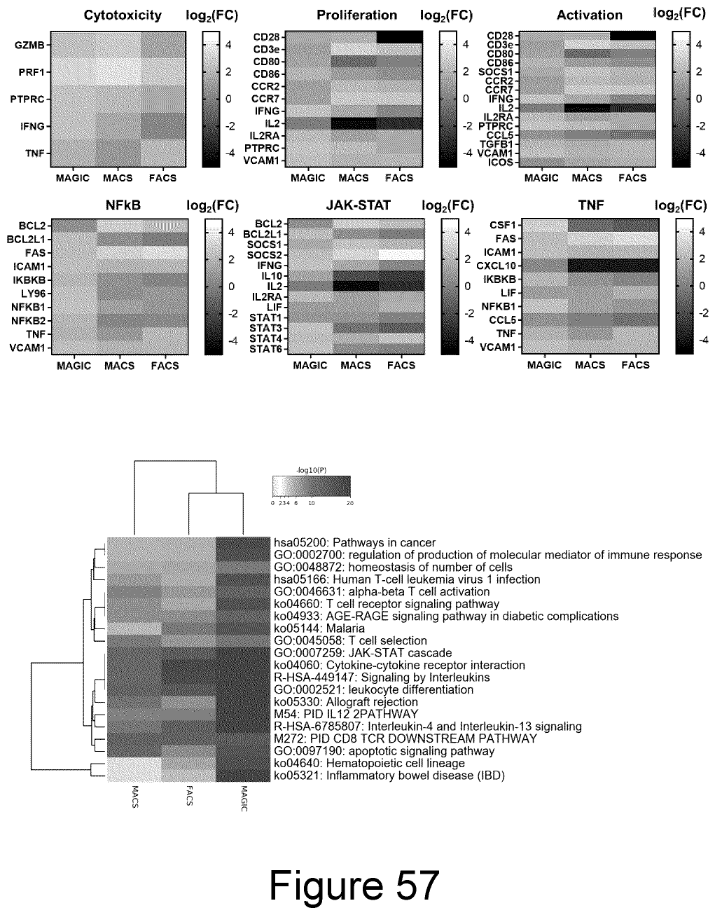

Subsequently, the cytotoxicity and reactivity of expanded TILs at the RNA and phenotypic levels was characterized. First, the relative expression of key immune pathways through qPCR was evaluated, using activated CD8⁺ splenocytes as the control (FIGS. 28 and 57). The majority of immune-responsive pathways and genes are upregulated, including NFkB, JAK-STAT, proliferation markers, and cytotoxic cytokines (FIG. 57). A pathway enrichment was further performed using the gene sets with log₂(FC)>1.5 in GO and KEGG databases (FIG. 29). MAGIC-TILs have the highest −log₁₀(P) in most of the pathways, suggesting an improved immune reactivity of MAGIC-TILs at the RNA level. An in vitro killing assay was constructed by co-culturing TILs with a monolayer of B16F10-OVA cells (FIG. 30). In agreement with the data collected at level of RNA, MAGIC-sorted TILs showed higher immune reactivity measured by the percentage of cell death at 24 and 48 hours. ELISA analysis of the supernatant during co-culture revealed that all types of TILs were functional. All types of TILs were capable of generating a cocktail of cytokines that promoted antigen presenting, cytotoxicity, T cell proliferation, monocyte recruitment, and anti-angiogenesis. Notably, MAGIC-TILs secreted a significantly higher level of IFNγ, which may explain their improved cytotoxicity over other TILs.

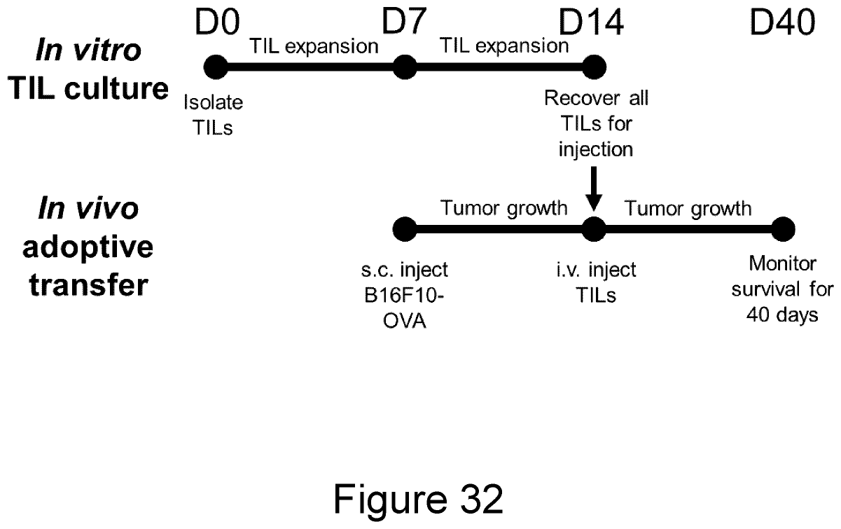

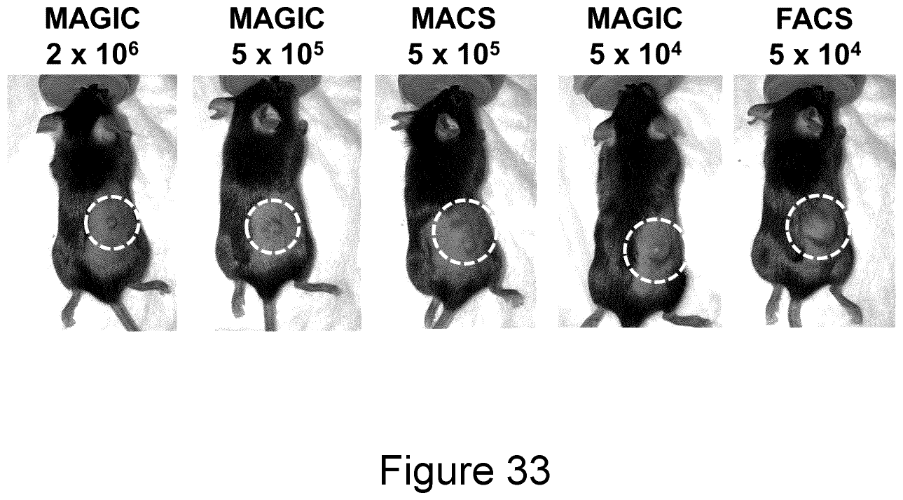

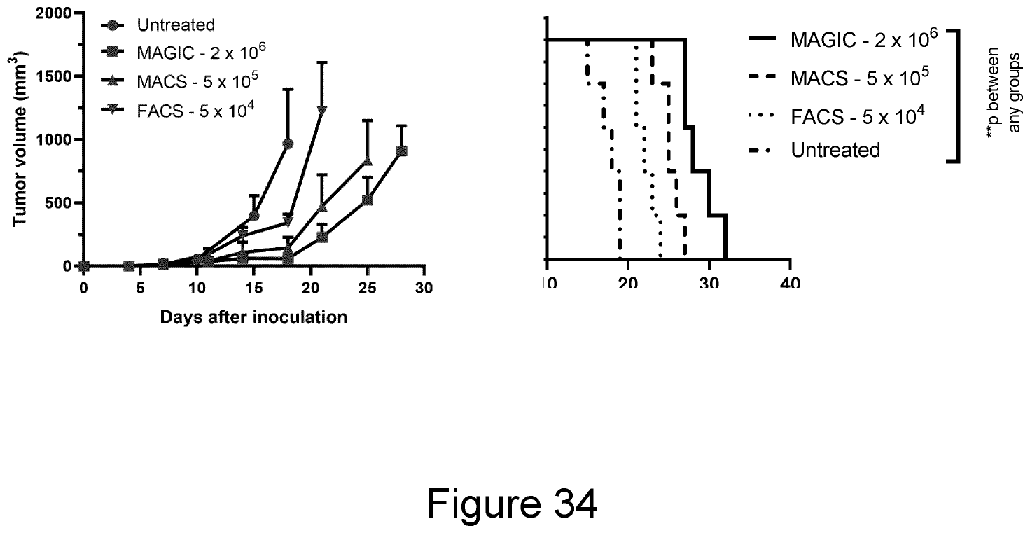

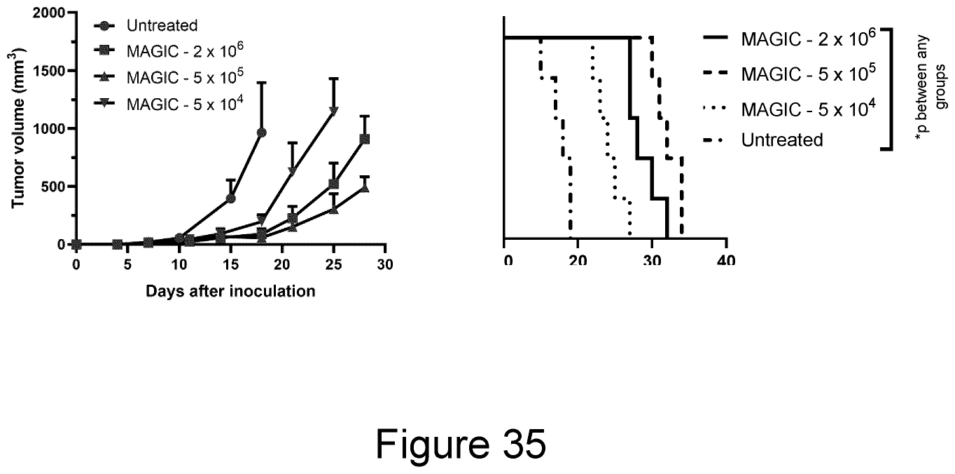

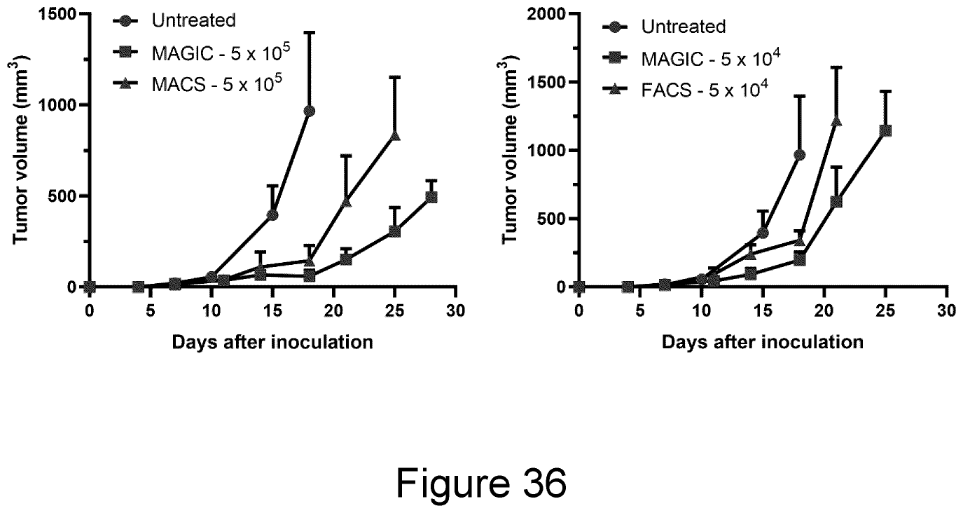

To further investigate the potency of TILs isolated by different approaches, two scenarios of animal studies were designed to systematically examine their therapeutic efficacy (see FIGS. 32 to 40). Scenario 1 has a fixed time point of injection (Day 7 after tumor introduction) with varied cell number, as a result of the different expansion curves of TILs (FIG. 32). It was observed that all TILs suppressed tumor growth compared to untreated control (FIGS. 33 and 34). The microfluidic-sorted TILs achieved the best therapeutic efficacy, possibly due to the high dosage number enabled by the rapid expansion, as well as the better cytotoxic phenotypes as a result of high diversity. The effects of dosage was also investigated for MAGIC-TILs. Interestingly, the dosage of 5×10⁵ offers statistically better median survival than the dosage of 2×10⁶ (32 vs 28 days, p=0.049). It is anticipate that the high dosage of TILs in one shot may result in some negative impacts on therapy.⁴⁰ Therefore, a dose near 5×10⁵ cells is the optimal dosage in the B16F10 model. In addition, the therapeutic outcomes of MAGIC/MACS-TILs was compared at the dose of 5×10⁵ cells (FIG. 36) and MAGIC/FACS-TILs at the dose of 5×10⁴ cells (FIG. 36). The results highlighted that the MAGIC-TILs were inherently more potent than MACS/FACS-TILs under the same dose.

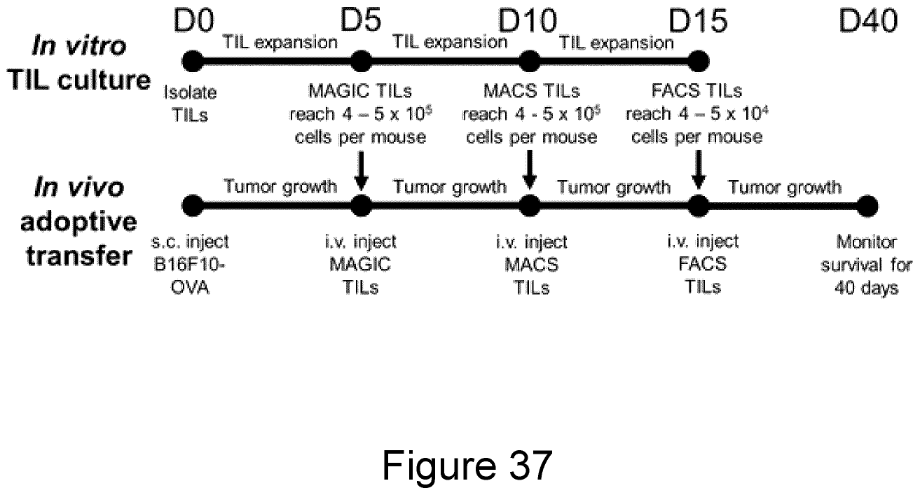

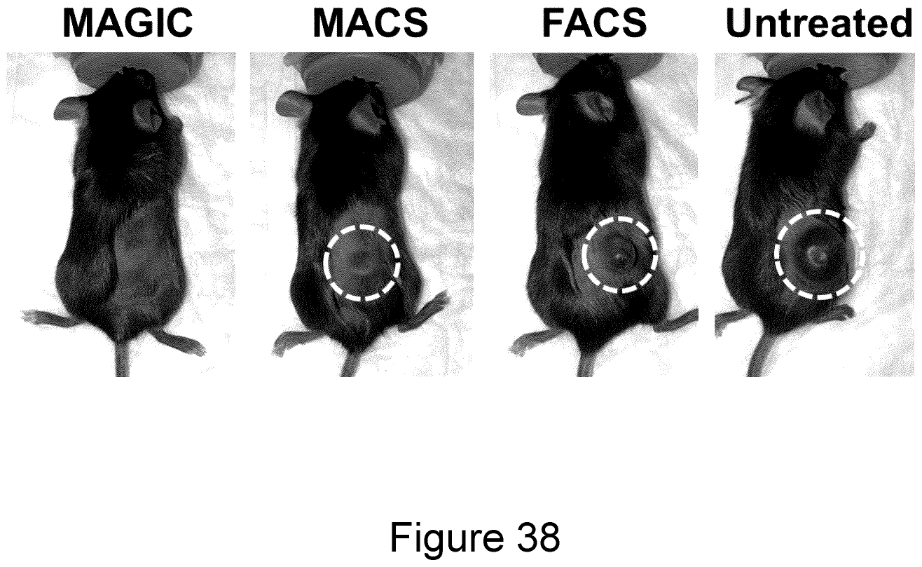

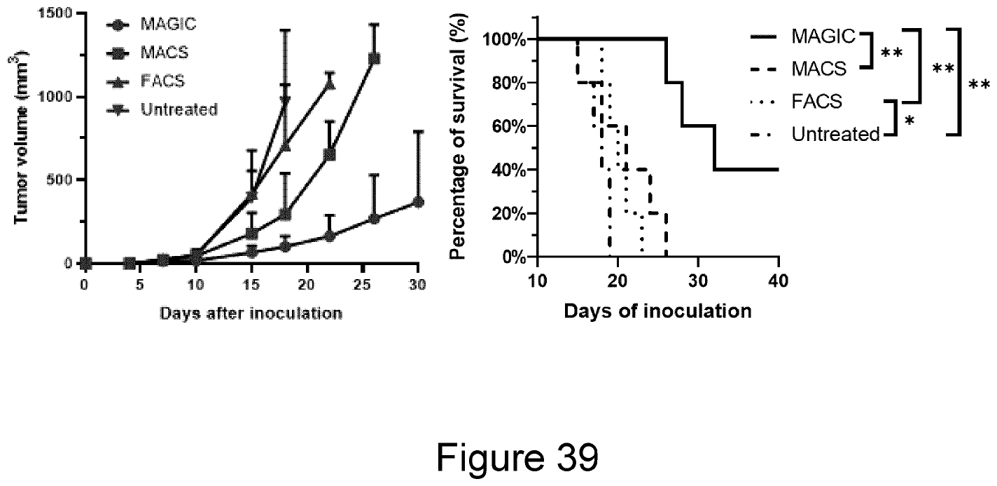

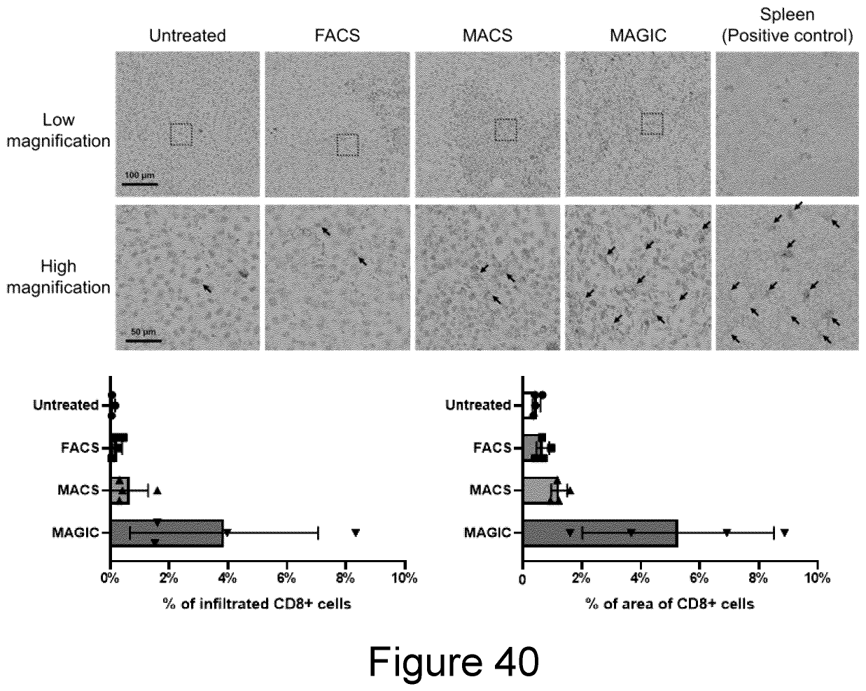

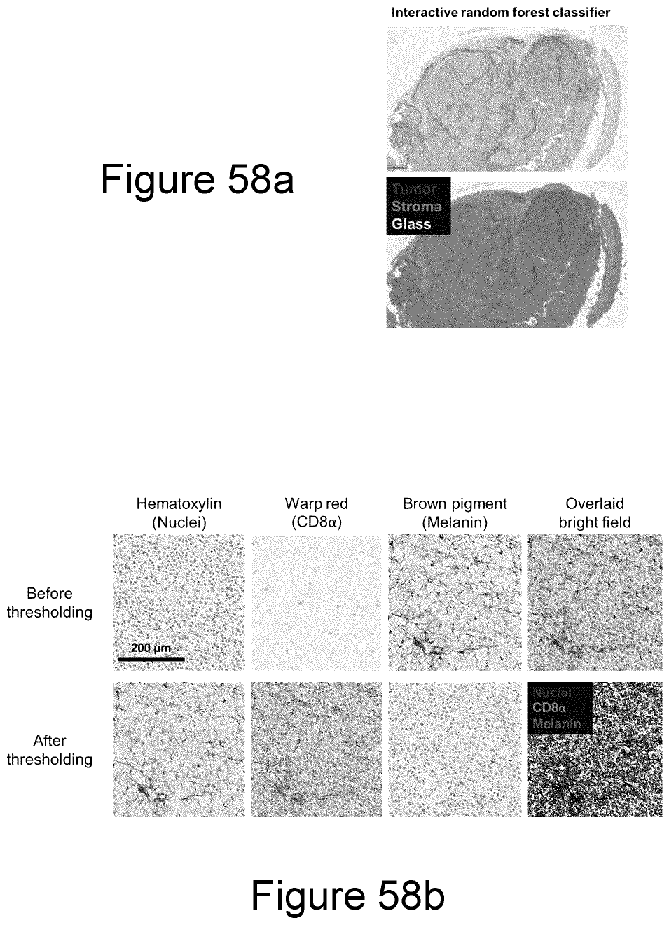

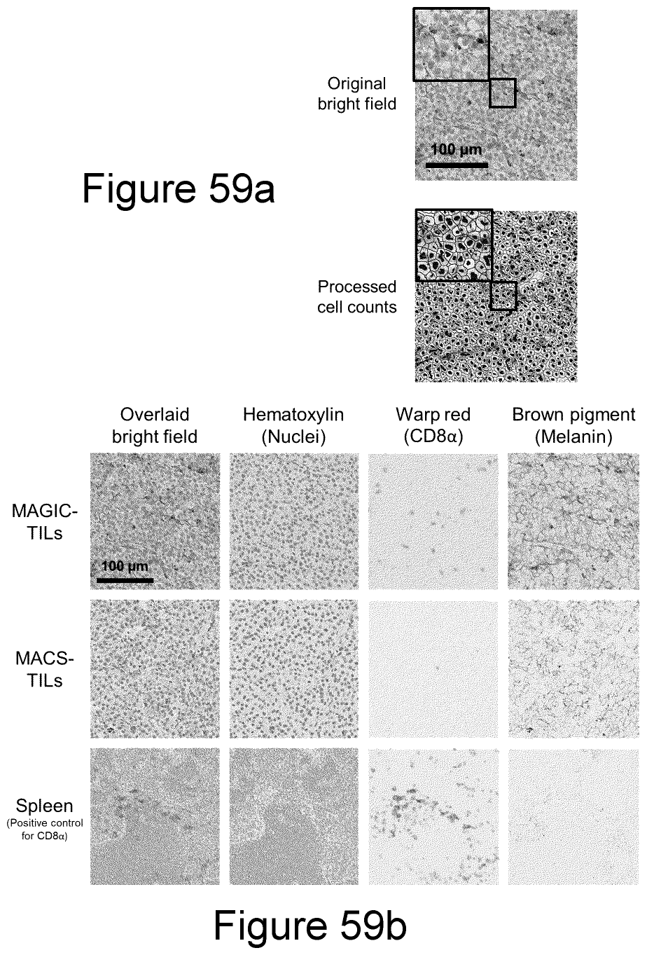

Next, the therapeutic outcomes of TILs was evaluated when isolation of TILs and the introduction of new tumors were performed on the same day (FIG. 37). This is to mimic the situation such that a patient has a rapidly growing metastasized tumor and deteriorated quickly, which represents 20% of the patients in clinical.⁹ TILs were injected once their quantity reaches the optimal dose (5×10⁵ cells). TILs were isolated using MAGIC/MACS/FACS-TIL on day 5, day 10, and day 15 at the optimal dose (except FACS as it failed to reach the target concentration). Under this setting, MAGIC-TILs still offer the best therapeutic outcomes as a result of high potency and early injection, likely due to the high initial recovery and rapid expansion in vitro (FIGS. 38 and 39). The median survival is 18, 20, 21, and 32 days for untreated, FACS, MACS, MAGIC-TILs, respectively. Interestingly, 2 out of 5 mice in the MAGIC-TILs group were in complete remission on day 40 as no visible tumor tissues were found subcutaneously. Such findings indicate that the MAGIC-TILs that underwent minimal in vitro expansion (5 days) may have better in vivo therapeutic efficacy. This result matches well with the idea of ‘young TILs’—minimally cultured TILs (referred to as young TILs) had a high level of antigen reactivity compared to the standard TILs that underwent prolonged expansion¹² and could effectively mediate the regression of metastatic melanoma.¹⁰ Besides, a recent study has highlighted that oligoclonal TILs are often outgrown by infrequent clonotypes with greater proliferative capacity.⁴¹ As a result, the clonal compositions and tumor reactivity of TILs are likely to change during in vitro expansion.⁴² This reveals the sophisticated nature of TIL expansion—enrichment of ‘right’ clonotypes is more desirable than the outgrowth of bulk TILs.⁴¹ Lastly, the number of infiltrated CD8⁺ T cells in tumors from scenario 2 was compared to directly benchmark their capability of infiltration (FIGS. 40a, 58 to 59b). The number and area of infiltrated CD8+ T cells (FIG. 40b) were upregulated 5-10 times higher in MAGIC groups, which is in good agreement with its positive therapeutic outcomes. Taken together, these discoveries culminate the importance of minimizing in vitro culture of TILs and directly prove the essentiality of having more initial TILs to start with through efficient cell sorting.

### Isolation of High-Potency TILs Using Quantitative MAGIC

Despite the great success of ACT, its objective response rate varies from 40-70%.⁸ Currently, it is unclear which phenotypes of TILs have the best therapeutic potency in vivo. Identification of highly tumor-reactive populations of TILs may lead to the better clinical success of ACT. Recent studies have highlighted the expression of CD39 may define highly potent TILs. On the one hand, CD39^{pos} populations, solely^{43,44} or in combination with other markers,⁴⁵ were reported to accurately determine the tumor-reactive populations of TILs and lead to improved clinical outcomes.⁴⁵ On the other hand, the expression of CD39 was highly associated with T cell exhaustion.⁴⁶ It is suggested that CD39^{neg} populations prompted long-term tumor management instead.⁴⁷ Hence, there is a lack of consensus regarding the role of CD39 expression in therapeutic efficacy. Interestingly, there is some evidence suggesting that the TILs with the medium expression of CD39 (CD39^{med}) retain inexhausted phenotype in vitro.⁴⁶ With the context that CD39^{neg} defines a bystander population⁴³ and CD39^{pos} defines an exhausted population,⁴⁷ it is hypothesized that CD39^{med}, a population that is disregarded by binary sorting and often subjectively assigned to positive⁴⁷ or negative⁴³ fractions, may possess improved therapeutic outcome.

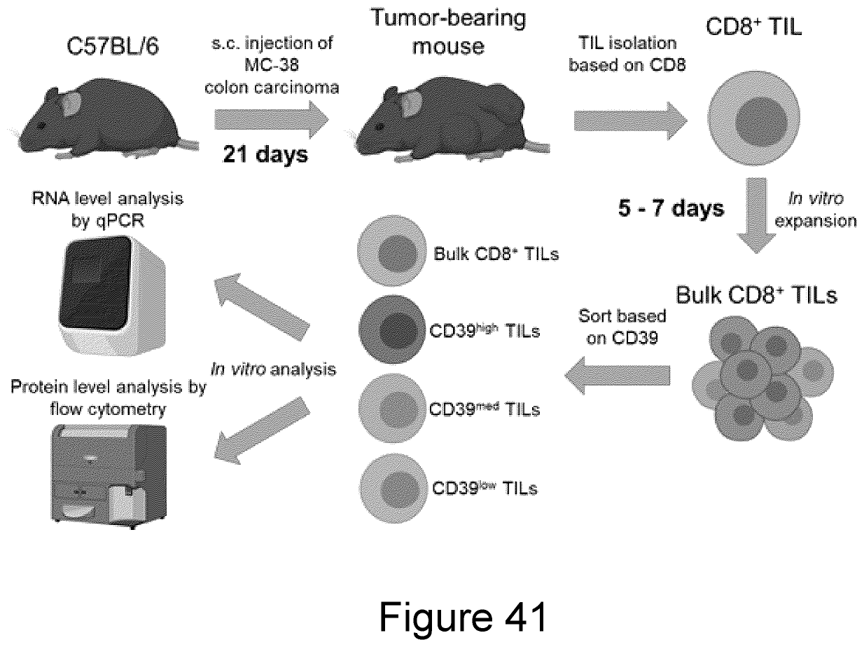

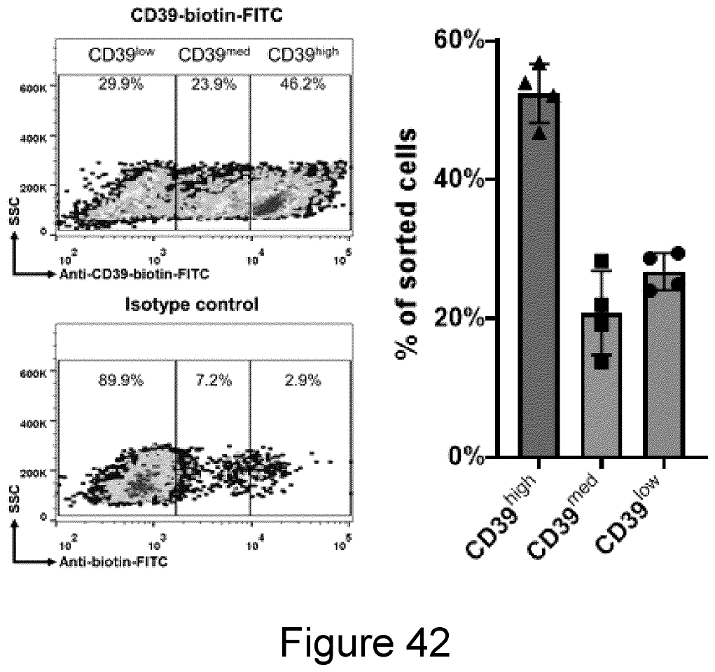

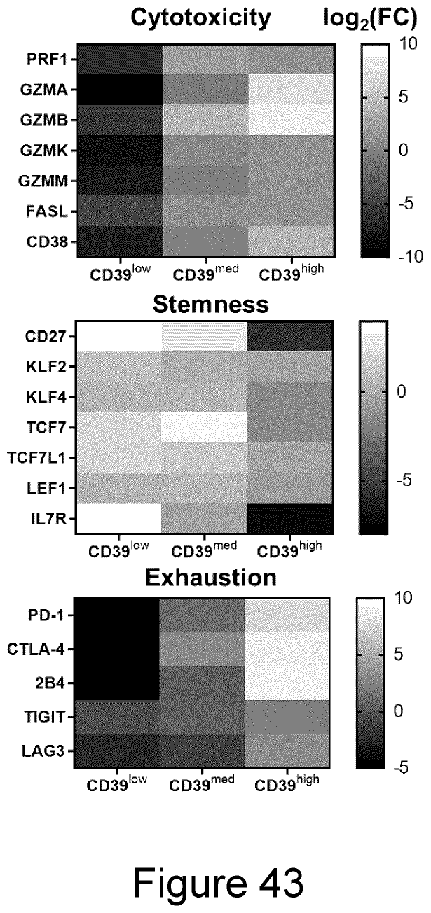

The experiments were initiated by the isolation of CD8+ T cells from a MC-38-bearing mouse model. The secondary quantitative (series system 100b) sorting based on CD39 was performed on day 5-7 post the CD8 isolation, followed by in vitro characterization (FIG. 41). It was first confirmed that the expression of CD39 within CD8+ T cells is not bimodal as about 25% of the cells are CD39^{med} (FIG. 42), which shares a similar profile to existing literature.⁴⁶ The flow rate of the quantitative MAGIC (qMAGIC) was finely tuned to 6 mL/hr and achieved a distinct separation among three CD39 populations (FIG. 42). The qPCR was next performed to analyze the phenotypes of different CD39 populations, including common markers of cytotoxicity, stemness, and exhaustion (FIG. 43). CD39^{low} population retained a low expression of cytotoxicity and exhaustion markers such as PRF1, GZMB, PD-1, CTLA-4, 2B4, and a high expression of stemness markers including CD27, TCF7, and IL7R, defining non-differentiated, stem-like phenotypes, which is in good agreement with the previous report.⁴⁷ On the contrary, CD39^{high} population defined a highly exhausted but cytotoxic phenotype that correlates well with existing articles.^{43,44} Interestingly, the CD39^{med} population had a balanced expression of cytotoxicity, stemness, and exhaustion markers. It is worth noting that CD39^{med} has the highest expression of TCF7, a memory stem-like T cell marker.

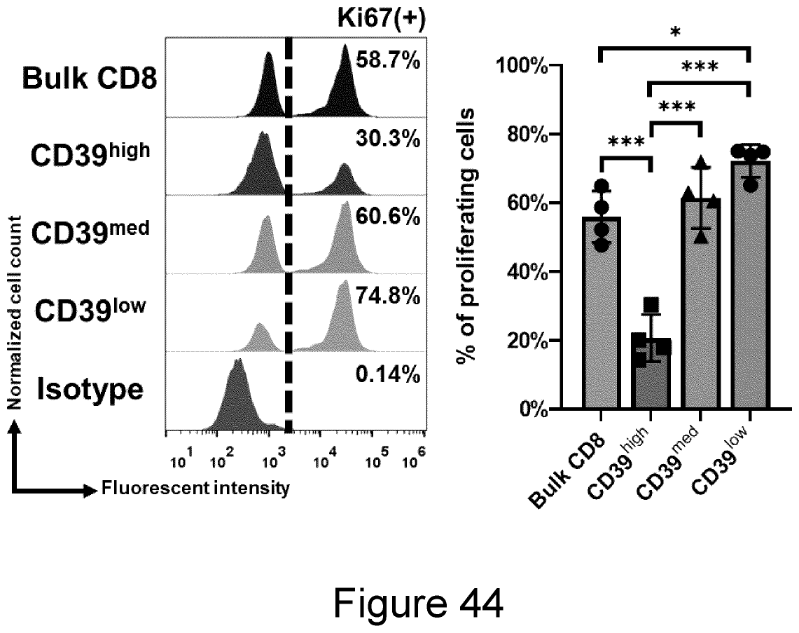

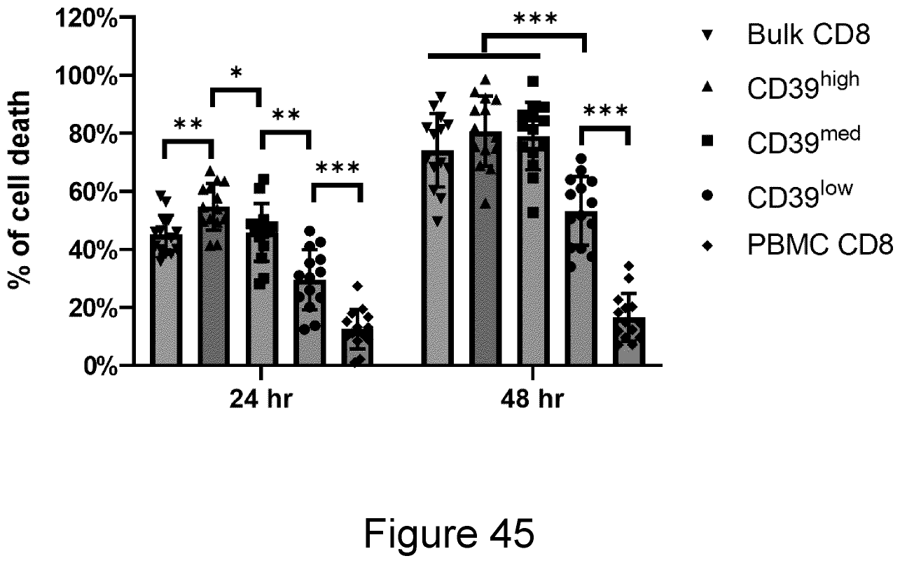

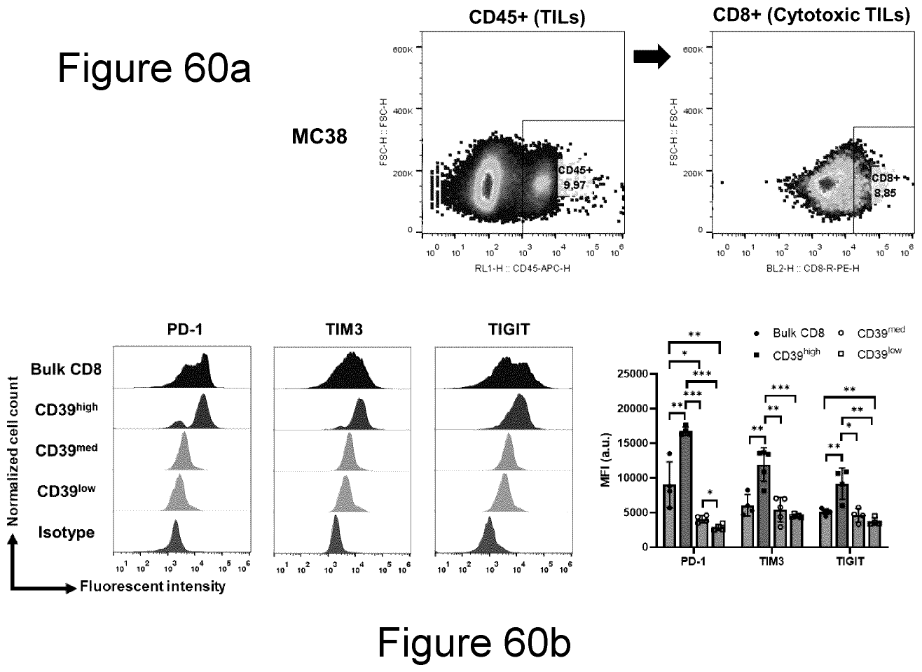

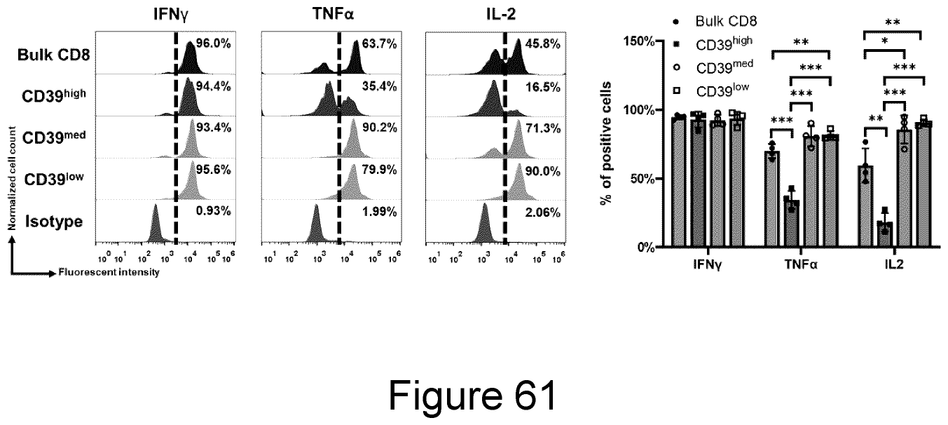

To verify the phenotypes at the protein level, intracellular flow cytometry was performed (FIGS. 44 and 60a to 61). A similar trend for each population was observed within the panel of exhaustion (PD-1, TIM3, and TIGIT) and stemness markers (CD27 and TCF7). Notably, it was found that the CD39^{high} population was with a significantly lower intracellular expression of TNF and IL-2, which is a sign of their exhausted, dysfunctional phenotypes. The dramatic decreasing of IL-2 also indicated such CD39^{high} population had diminished capability of proliferation. This hypothesis is confirmed by the Ki67 staining (FIG. 5F). The percentage of memory stem-like T cells was verified with each population through the staining of CCR7/CD45 (FIG. 45) and found most of the stem-like T cells were presented in CD39^{low} and CD39^{med} populations. Also, it was observed that the CD39^{med} population has the highest percentage of PD-1⁺/TCF7⁺ cells. As such population has been reported as a stem-like T cell progenitor with defined tumor specificity,⁴⁸ it was concluded that CD39^{med} population is a distinct population that largely contains a stem-like T cell progenitor population.

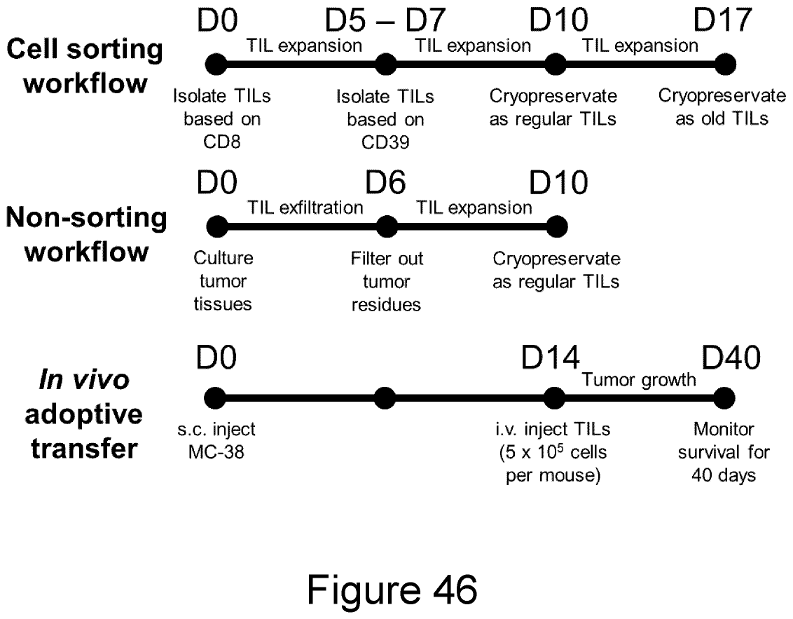

Next, the in vitro co-culture killing assay was performed as a direct measurement of cytotoxicity at the functional level (FIG. 46). It was noticed that the CD39^{high} population showed the highest tumor-killing activities 24 hours posting seeding. CD39^{med} population had slightly lower efficacy in the first 24 hours but were able to elevate its killing activities to the same level of CD39^{high} population in 48 hours. Therefore, the PD-1⁺/TCF7⁺/CD39^{med} population could rapidly generate a highly cytotoxic population for effective tumor killing. CD39^{low} population, however, retained significantly lower killing activities in 24 and 48 hours, meaning CD39^{low} cells may lack tumor reactivity or have limited ability to generate cytotoxic population. This finding is in good agreement with the previous studies reporting such populations to lack PD-1 expression⁴⁶ and contain non-specific bystander cells.⁴³

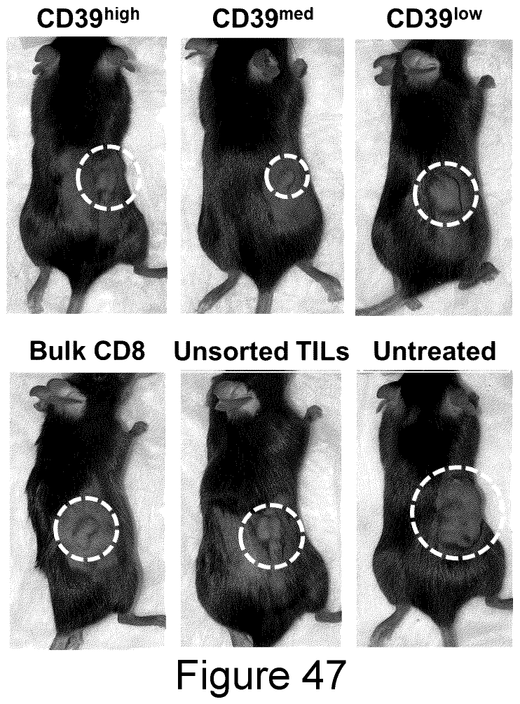

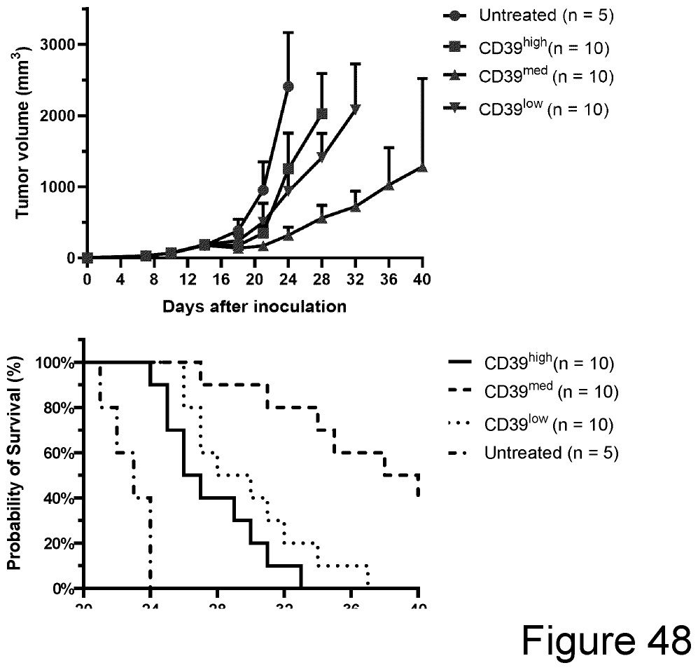

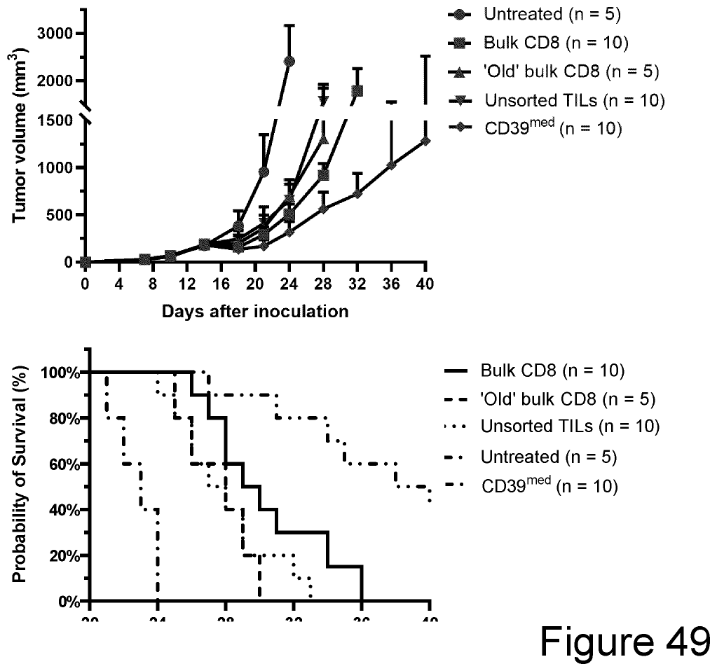

The anti-tumor efficacy of different CD39 populations in vivo was examined by reintroducing the sorted TILs into MC-38-bearing mice (FIGS. 46 and 47). CD39^{med} TILs showed the best long-term anti-tumor activity among all CD39 populations, extended the median survival by 60% compared to the untreated group (FIG. 48). Interestingly, it was found CD39^{high} TILs were highly effective in the beginning but failed to manage tumor growth after one week of injection. This is possibly due to its non-renewable, terminated phenotypes. A side-by-side comparison of CD39^{med} TILs was performed among other TIL types, including the bulk CD8 TILs that underwent minimal (‘young TILs’) and longer in vitro culture (‘Old TILs’), and unsorted TILs that exfiltrated from segmented tumor fractions. Judged from tumor volume and median survival, CD39^{med} TILs offered the best therapeutic efficacy among all TIL types (FIG. 49).

### Sorting Circulating TILs

The disclosed systems and methods are applicable to other cell types in different body fluids, such as rare tumor-reactive T cells in peripheral blood. In addition, the disclosed systems and methods can also sort cells based on not only the level of expression of a specific protein, but also the reactivity of T cell receptor (TCR).

The disclosed systems and methods are also applicable to various types of sorting applications, such as positive, negative, or quantitative sorting. In the example described below, a protocol was developed to sort circulating tumor-reactive T cells from peripheral blood through a two-step procedure. Firstly, a negative selection of CD8 cells from RBC-lysed blood was performed, followed by a multimer-based positive to isolate high-purity T cells with reactivity to specific antigens (also referred to as tumor/antigen-specific T cells) from peripheral blood.

Animal models was first established with two defined highly immunogenic epitopes—chicken ovalbumin (OVA₂₅₇₋₂₆₄, SIINFEKL) in C57BL6 model and influenza A hemagglutinin (HA₅₃₃₋₅₄₁, IYSTVASSL) in Balb/c model. Tumor cells with the expression of epitopes were injected subcutaneously. The immunogenicity of these epitopes led to the generation of OVA/HA-reactive T cells in blood. These rare reactive T cells can be recognized through MHC (Major histocompatibility complex) or HLA (human leukocyte antigen)-specific multimers. The multimers can be further magnetically labeled with MNPs to allow the capture of tumor/epitope-reactive T cells.

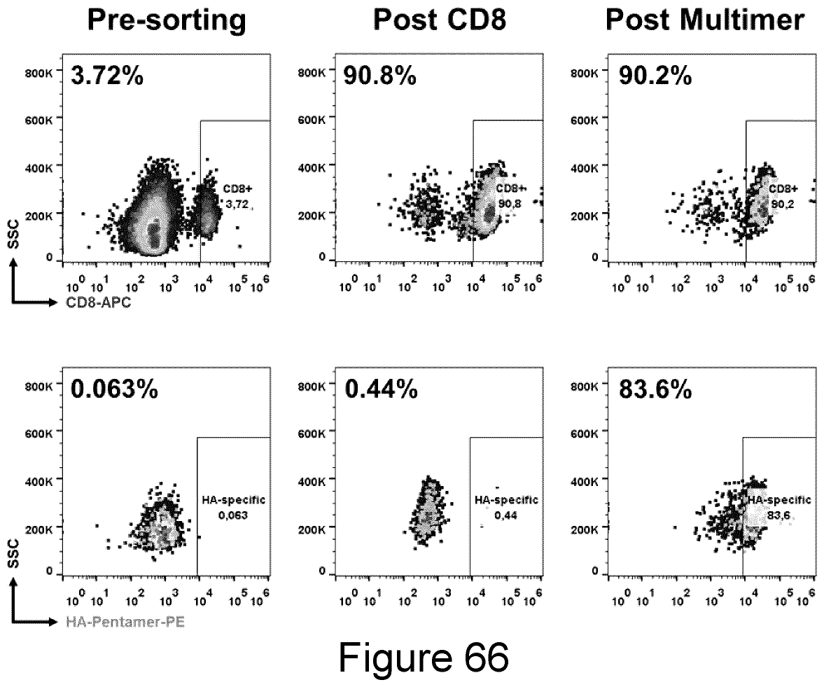

It is worth noting that before isolation, the abundancy of HA-reactive T cells only represents 0.063% of the mononuclear cell population (see FIG. 66, pre-sorting panel). During isolation, mouse whole blood was collected at the mid-late stage of tumor development. Red blood cells (RBC) were lysed by RBC lysis buffer. The lysed cells were labeled by a cocktail targeting other portions of blood cells, including CD19 for B cells, CD11b for monocytes, etc. Such a cocktail can be prepared in-house or purchased directly from commercially available resources (e.g. untouched mouse CD8 cells kit, cat #11417D, Thermo Fisher). Cocktail-labeled cells were subsequently labeled by MNPs and sorted by the proposed systems at the flow rate of 8-32 mL/hr (in particular, 16 mL/hr for the untouched mouse CD8 cells kit described above was used). After the negative selection of CD8, the purity of CD8+ cells was improved from 3.7% to 90.8% (FIG. 66, post-CD8 panel) and the rarity of HA-reactive T cells was promoted from 0.063% to 0.44% (FIG. 66, post-CD8 panel).

The positive selection was performed next based on multimer to purify HA-reactive T cells from bulk CD8+ populations. Bulk CD8⁺ T cells were labeled by corresponding PE-conjugated multimers (pentamer in this particular case) and anti-PE MNPs accordingly. Conjugation of the fluorophore on multimers is flexible—FITC (fluorescein isothiocyanate), PE (phycoerythrin), APC (Allophycocyanin), Cyanine families, and biotin would all work using the corresponding MNPs for labeling (e.g. anti-biotin MNPs). Multimer-labeled cells were sorted using the proposed system at the flow rate of 2-8 mL/hr (4 mL/hr in this specific case). After the positive selection, the purity of HA-reactive T cells was improved from 0.44% to 83.6% (FIG. 66, post multimer panel)

Although the present disclosure describes the disclosed methods and devices for TIL sorting and recovery, the disclosed methods and devices may be used for magnetic profiling of other particles, including other cells, for other cell therapy purposes. For example, the disclosed devices, systems, and methods may also be used to recover target cells from other fluid sources such as peripheral blood, vascularized tumors, malignant pleural effusion, lymphatic fluid, the fluid portion of bone marrow, and other cell-carrying fluids.

The embodiments of the present disclosure described above are intended to be examples only. The present disclosure may be embodied in other specific forms. Alterations, modifications and variations to the disclosure may be made without departing from the intended scope of the present disclosure. While the systems, devices and processes disclosed and shown herein may comprise a specific number of elements/components, the systems, devices and assemblies could be modified to include additional or fewer of such elements/components. For example, while any of the elements/components disclosed may be referenced as being singular, the embodiments disclosed herein could be modified to include a plurality of such elements/components. Selected features from one or more of the above-described embodiments may be combined to create alternative embodiments not explicitly described. All values and sub-ranges within disclosed ranges are also disclosed. The subject matter described herein intends to cover and embrace all suitable changes in technology. All references mentioned are hereby incorporated by reference in their entirety.

### REFERENCES

1. Gajewski, T. F., Schreiber, H. & Fu, Y. X. Innate and adaptive immune cells in the tumor microenvironment. Nat. Immunol. 14, 1014-1022 (2013).
2. Mellman, I., Coukos, G. & Dranoff, G. Cancer immunotherapy comes of age. Nature 480, 480-489 (2011).
3. Rohaan, M. W., Van Den Berg, J. H., Kvistborg, P. & Haanen, J. B. A. G. Adoptive transfer of tumor-infiltrating lymphocytes in melanoma: A viable treatment option 11 Medical and Health Sciences 1107 Immunology 11 Medical and Health Sciences 1112 Oncology and Carcinogenesis. J. Immunother. Cancer 6, 1-16 (2018).
4. Seliktar-Ofir, S. et al. Selection of shared and neoantigen-reactive T cells for adoptive cell therapy based on CD137 separation. Front. Immunol. 8, 1-14 (2017).
5. Lee, H. J. et al. Expansion of tumor-infiltrating lymphocytes and their potential for application as adoptive cell transfer therapy in human breast cancer. Oncotarget 8, 113345-113359 (2017).
6. Hurtado, M. O. et al. Tumor infiltrating lymphocytes expanded from pediatric neuroblastoma display heterogeneity of phenotype and function. PLoS One 14, 1-18 (2019).
7. Lindenberg, M. A. et al. Treatment with Tumor-infiltrating Lymphocytes in Advanced Melanoma Evaluation of Early Clinical Implementation of an Advanced Therapy Medicinal Product. J. Immunother. 41, 413-425 (2018).
8. Lindenberg, M. et al. Evaluating different adoption scenarios for TIL-therapy and the influence on its (early) cost-effectiveness. BMC Cancer 20, 1-14 (2020).
9. Borch, T. H. et al. Future role for adoptive T-cell therapy in checkpoint inhibitor-resistant metastatic melanoma. J. Immunother. Cancer 8, 1-7 (2020).
10. Dudley, M. E. et al. CD8+ enriched ‘Young’ tumor infiltrating lymphocytes can mediate regression of metastatic melanoma. Clin. Cancer Res. 16, 6122-6131 (2010).
11. Donia, M. et al. Characterization and comparison of ‘standard’ and ‘young’ tumour-infiltrating lymphocytes for adoptive cell therapy at a danish translational research institution. Scand. J. Immunol. 75, 157-167 (2012).
12. Tran, K. Q. et al. Minimally Cultured Tumor-infiltrating Lymphocytes Display Optimal Characteristics for Adoptive Cell Therapy. J. Immunother. 31, 742-751 (2008).
13. Forget, M. A. et al. A novel method to generate and expand clinical-grade, genetically modified, tumor-infiltrating lymphocytes. Front. Immunol. 8, 1-8 (2017).
14. Wickström, S. & Lövgren, T. Expansion of tumor-infiltrating lymphocytes from melanoma tumors. Methods in Molecular Biology vol. 1913 105-118 (2019).
15. Prieto, P. A., Durflinger, K. H., Wunderlich, J. R., Rosenberg, S. A. & Dudley, M. E. Enrichment of CD8+ Cells From Melanoma Tumor-infiltrating Lymphocyte Cultures Reveals Tumor Reactivity for Use in Adoptive Cell Therapy. J. Immunother. 33, 547-556 (2010).
16. Schober, K. & Busch, D. H. TIL 2.0: More effective and predictive T-cell products by enrichment for defined antigen specificities. Eur. J. Immunol. 46, 1335-1339 (2016).
17. Andersen, R. et al. Long-Lasting complete responses in patients with metastatic melanoma after adoptive cell therapy with tumor-infiltrating lymphocytes and an attenuated il2 regimen. Clin. Cancer Res. 22, 3734-3745 (2016).
18. Dong, M. B. et al. Systematic Immunotherapy Target Discovery Using Genome-Scale In Vivo CRISPR Screens in CD8 T Cells. Cell 178, 1189-1204.e23 (2019).
19. Hall, M. L. et al. Expansion of tumor-infiltrating lymphocytes (TIL) from human pancreatic tumors. J. Immunother. Cancer 4, 1-12 (2016).
20. Garaud, S. et al. Tumor-infiltrating B cells signal functional humoral immune responses in breast cancer. JCI Insight 4, 1-20 (2019).
21. Wagner, P. et al. Detection and functional analysis of tumor infiltrating T-lymphocytes (TIL) in liver metastases from colorectal cancer. Ann. Surg. Oncol. 15, 2310-2317 (2008).
22. Salot, S. et al. Large scale expansion of Vγ9Vδ2 T lymphocytes from human peripheral blood mononuclear cells after a positive selection using MACS ‘TCR γ/δ+T cell isolation kit’. J. Immunol. Methods 347, 12-18 (2009).
23. Faraghat, S. A. et al. High-Throughput, low-loss, low-cost, and label-free cell separation using electrophysiology-Activated cell enrichment. Proc. Natl. Acad. Sci. U.S.A. 114, 4591-4596 (2017).
24. Sutermaster, B. A. & Darling, E. M. Considerations for high-yield, high-throughput cell enrichment: fluorescence versus magnetic sorting. Sci. Rep. 9, 1-9 (2019).
25. Mair, B. et al. High-throughput genome-wide phenotypic screening via immunomagnetic cell sorting. Nat. Biomed. Eng. 3, 796-805 (2019).
26. Moore, D. K. et al. Isolation of B-cells using Miltenyi MACS bead isolation kits. PLoS One 14, 1-15 (2019).
27. Geens, M. et al. The efficiency of magnetic-activated cell sorting and fluorescence-activated cell sorting in the decontamination of testicular cell suspensions in cancer patients. Hum. Reprod. 22, 733-742 (2007).
28. Wyatt Shields Iv, C., Reyes, C. D. & López, G. P. Microfluidic cell sorting: A review of the advances in the separation of cells from debulking to rare cell isolation. Lab Chip 15, 1230-1249 (2015).
29. Lee, W., Tseng, P. & Carlo, D. Di. Microtechnology for Cell Manipulation and Sorting. Microtechnology for Cell Manipulation and Sorting (2017). doi :10.1007/978-3-319-44139-9.
30. Lin, E. et al. High-Throughput Microfluidic Labyrinth for the Label-free Isolation of Circulating Tumor Cells. Cell Syst. 5, 295-304.e4 (2017).
31. Chen, K. et al. Integration of Lateral Filter Arrays with Immunoaffinity for Circulating-Tumor-Cell Isolation. Angew. Chem., Int. Ed. 58, 7606-7610 (2019).
32. Murray, C., Pao, E., Jann, A., Park, D. E. & Di Carlo, D. Continuous and Quantitative Purification of T-Cell Subsets for Cell Therapy Manufacturing Using Magnetic Ratcheting Cytometry. SLAS Technol. 23, 326-337 (2018).
33. Murray, C. et al. Unsupervised capture and profiling of rare immune cells using multi-directional magnetic ratcheting. Lab Chip 18, 2396-2409 (2018).
34. Wang, Z. et al. Ultrasensitive and rapid quantification of rare tumorigenic stem cells in hPSC-derived cardiomyocyte populations. Sci. Adv. 6, 1-12 (2020).
35. Mohamadi, R. M. et al. Nanoparticle-mediated binning and profiling of heterogeneous circulating tumor cell subpopulations. Angew. Chem., Int. Ed. 54, 139-143 (2015).
36. Labib, M. et al. Tracking the expression of therapeutic protein targets in rare cells by antibody-mediated nanoparticle labelling and magnetic sorting. Nat. Biomed. Eng. (2020) doi:10.1038/s41551-020-0590-1.
37. Allen, B. M. et al. Systemic dysfunction and plasticity of the immune macroenvironment in cancer models. Nat. Med. 26, 1125-1134 (2020).
38. Del Monte, U. Does the cell number 109 still really fit one gram of tumor tissue? Cell Cycle 8, 505-506 (2009).
39. Ma, Q., Wang, Y., Lo, A. S. Y., Gomes, E. M. & Junghans, R. P. Cell density plays a critical role in ex vivo expansion of T cells for adoptive immunotherapy. J. Biomed. Biotechnol. 2010, (2010).
40. Schmidts, A., Wehrli, M. & Maus, M. V. Toward Better Understanding and Management of CAR-T Cell-Associated Toxicity. Annu. Rev. Med. 72, 1-18 (2021).
41. Poschke, I. C. et al. The Outcome of Ex Vivo TIL Expansion Is Highly Influenced by Spatial Heterogeneity of the Tumor T-Cell Repertoire and Differences in Intrinsic In Vitro Growth Capacity between T-Cell Clones. Clin. Cancer Res. 26, 4289-4301 (2020).
42. Lozano-Rabella, M. & Gros, A. TCR Repertoire Changes during TIL Expansion: Clonal Selection or Drifting? Clin. Cancer Res. 26, 4177-4179 (2020).
43. Simoni, Y. et al. Bystander CD8+ T cells are abundant and phenotypically distinct in human tumour infiltrates. Nature 557, 575-579 (2018).
44. Kortekaas, K. E. et al. CD39 Identifies the CD4+ Tumor-Specific T-cell Population in Human Cancer. Cancer Immunol. Res. 8, 1311-1321 (2020).
45. Duhen, T. et al. Co-expression of CD39 and CD103 identifies tumor-reactive CD8 T cells in human solid tumors. Nat. Commun. 9, (2018).
46. Canale, F. P. et al. CD39 expression defines cell exhaustion in tumor-infiltrating CD8+ T cells. Cancer Res. 78, 115-128 (2018).
47. Krishna, S. et al. Stem-like CD8 T cells mediate response of adoptive cell immunotherapy against human cancer. Science 370, 1328-1334 (2020).
48. Siddiqui, I. et al. Intratumoral Tcf1+PD-1+CD8+T Cells with Stem-like Properties Promote Tumor Control in Response to Vaccination and Checkpoint Blockade Immunotherapy. Immunity 50, 195-211.e10 (2019).
49. Venzac, B. et al. PDMS Curing Inhibition on 3D-Printed Molds: Why? Also, How to Avoid It?. Anal. Chem. 93, 19, 7180-7187 (2021).

1. A device for recovering magnetically tagged target cells from a fluid collection of cells, the device comprising:
a magnet plate having an array of micro-magnets positioned therein, the array of micro-magnets producing a magnetic field along the magnet plate;
a modular chip releasably coupled in covering relation to the magnet plate, the modular chip having:
a flow chamber with an inlet and an outlet; and
a plurality of flow rate-reducing structures in the flow chamber, each structure comprising a trapping surface shaped to reduce flow rate in a vicinity of the trapping surface, the magnetic field produced by the array of micro-magnets cooperating with the flow rate-reducing structures to define a respective capture zone in the vicinity of each of the flow rate-reducing structures;
wherein the magnetic field, in the capture zone, is sufficiently high to overcome drag force on the target cells to promote capture of the target cells, from the collection of cells, in the capture zone; and
wherein separation of the modular chip from the magnet plate allows for separation and recovery of the captured target cells from the modular chip.

2. The device of claim 1, wherein the modular chip comprises a glass base and chamber walls, the chamber walls and glass base collectively forming the flow chamber, the chamber walls and the plurality of flow rate-reducing structures extending from the glass base.

3. The device of claim 2, wherein the chamber walls and the plurality of flow rate-reducing structures are made of PDMS.

4. The device of claim 2 or 3, wherein the glass base is between 0.05 and 0.5 mm thick.

5. The device of claim 4, wherein the glass base is less than 0.1 mm thick.

6. The device of any of claims 2 to 5, wherein the glass base is coated with PDMS.

7. The device of any of claims 2 to 6, wherein the glass base is 75 mm long and 50 mm wide.

8. The device of any of claims 1 to 7, wherein height of each of the plurality of flow rate-reducing structures is between 50 microns and 800 microns.

9. The device of claim 8, wherein the height of each of the plurality of flow rate-reducing structures is 800 microns where the laminar flow is maintained.

10. A system for recovering magnetically tagged target cells from a collection of cells, the system comprising:
a magnet plate having an array of micro-magnets positioned therein, the array of micro-magnets producing a magnetic field along the magnet plate;
one or more modular chips releasably coupled in covering relation to the magnet plate, each of the one or more modular chips having:
a flow chamber with an inlet and an outlet; and
a plurality of flow rate-reducing structures in the flow chamber, each structure comprising a trapping surface shaped to reduce flow rate in a vicinity of the trapping surface, the magnetic field produced by the array of micro-magnets cooperating with the flow rate-reducing structures to define a respective capture zone in the vicinity of each of the flow rate-reducing structures;
wherein the magnetic field, in the capture zone, is sufficiently high to overcome drag force on the target cells to promote capture of the target cells, from the collection of cells, in the capture zone; and
wherein separation of the modular chip from the magnet plate allows for separation and recovery of the captured target cells from the modular chip; and
a scaffold configured to retain the magnet plate.

11. The system of claim 10, wherein the magnet plate is integrated into the scaffold.

12. The system of claim 10, wherein the magnet plate is releasably securable to the scaffold.

13. The system of any of claims 10 to 12, wherein the one or more modular chips comprise a first modular chip and a second modular chip.

14. The system of claim 13, wherein the first modular chip is releasably coupled in covering relation to a first portion of the magnet plate, and the second modular chip is releasably coupled in covering relation to a second portion of the magnet plate.

15. The system of claim 13, wherein the magnet plate comprises a first magnet plate and a second magnet plate, the first modular chip being releasably coupled in covering relation to the first magnet plate, and the second modular chip being releasably coupled in covering relation to the second magnet plate.

16. The system of claim 15, wherein the first and second magnet plates are positioned in parallel in the scaffold.

17. The system of any of claims 13 to 16, wherein the scaffold further comprises a first connector positioned proximate an end of each of the first and second modular chips, and a second connector positioned proximate another end of each of the first and second modular chips, the first and second connectors being fluidly coupleable to the corresponding inlet and outlet of each corresponding first or second modular chip.

18. The system of any of claims 13 to 17, wherein the first and second modular chips are fluidly connected in parallel to a source of the collection of cells.

19. The system of any of claims 13 to 17, wherein the first and second modular chips are fluidly connected in series, the outlet of the first modular chip being in direct fluid communication with the inlet of the second modular chip.

20. The system of any of claims 13 to 19, wherein the height of the plurality of flow rate-reducing structures in each of the first and second modular chips is the same.

21. The system of claim 20, wherein the height of the plurality of flow rate-reducing structures in each of the first and second modular chips is 100 microns.

22. The system of any of claims 13 to 19, wherein the height of the plurality of flow rate-reducing structures in the first modular chip is different from the height of the plurality of flow rate-reducing structures in the second modular chip.

23. The system of claim 22, wherein the height of the plurality of flow rate-reducing structures in the first modular chip is lower than the height of the plurality of flow rate-reducing structures in the second modular chip.

24. The system of any of claims 13 to 23, wherein the one or more modular chips further comprises:
a third modular chip; and
the third modular chip is releasably coupled in covering relation to a third portion of the magnet plate.

25. A method for recovering magnetically tagged target cells from a fluid collection of cells, the method comprising:
introducing the fluid collection of cells containing the magnetically tagged target cells into a device comprising a magnet plate having an array of micro-magnets positioned therein, the array of micro-magnets producing a magnetic field along the magnet plate, and a modular chip releasably coupled in covering relation to the magnet plate, the modular chip having a flow chamber with a plurality of flow rate-reducing structures, the magnetically tagged target cells being susceptible to a magnetic attraction force and being trapped by the flow rate-reducing structures as they travel through the flow chamber;
washing non-target cells out of the device;
separating the modular chip from the magnet plate; and
recovering the magnetically tagged target cells from the modular chip.

26. The method of claim 25, further comprising:
dissolving a tumour sample from a patient into single cells in the fluid collection of cells; and
labelling the single cells with magnetic particles prior to the introducing.

27. The method of claim 25, wherein the fluid collection of cells is from peripheral blood, vascularized tumors, malignant pleural effusion, lymphatic fluid, a fluid portion of bone marrow, or another cell-carrying fluid.

28. The method of any of claims 25 to 27, wherein the fluid collection of cells is introduced into the flow chamber of the device at a flow rate between 6-32 mL/hr.

29. The method of claim 28, wherein the flow rate is 16 mL/hr.

30. The method of claim 28, wherein the flow rate is 32 mL/hr.

31. The method of any of claims 25 to 30, wherein the washing comprising introducing a flushing fluid into the device with a syringe.

32. A method for recovering magnetically tagged target cells from a fluid collection of cells, the method comprising:
introducing the fluid collection of cells containing the magnetically tagged target cells into the system of claim 19 through the inlet of the first modular chip, directing the fluid sample from the outlet of the first modular chip into the inlet of the second modular chip;
washing non-target cells out of the system;
separating the first and second modular chips from the magnet plate; and
recovering the magnetically tagged target cells from the first and second modular chips.

33. A method for recovering magnetically tagged target cells from a fluid collection of cells, the method comprising:
introducing the fluid collection of cells containing the magnetically tagged target cells into the system of claim 18 through the inlet of the first modular chip and the inlet of the second modular chip;
washing non-target cells out of the system;
separating the first and second modular chips from the magnet plate; and
recovering the magnetically tagged target cells from the first and second modular chips.
